(19) United States
(12) Patent Application Publication
(10) Pub. No.: US 2026/0325727 A1
(43) Pub. Date: Oct. 22, 2026

(54) EXTRACORPOREAL BLOOD TREATMENT SYSTEM

(71) Applicant: NextKidney SA, Lausanne (CH)

(72) Inventors: Ricardo ALLENDES, Lausanne (CH); Yannick SUBLET, Lausanne (CH); Christian BLUCHEL, Lausanne (CH); Joël REY, Lausanne (CH); Anaïs VECTEN, Lausanne (CH); Christophe CONAN, Lausanne (CH)

(21) Appl. No.: 19/123,015

(22) PCT Filed: Oct. 31, 2023
(86) PCT No.: PCT/IB2023/060957

§ 371 Date: Apr. 21, 2025

(30) Foreign Application Priority Data
Nov. 2, 2022 (EP) 22205023.9
Nov. 2, 2022 (EP) 22205030.4
Nov. 2, 2022 (EP) 22205036.1
Nov. 2, 2022 (EP) 22205040.3
Nov. 2, 2022 (EP) 22205043.7

(51) Int. Cl. A61M 1/14; A61J 1/05

(52) U.S. Cl. CPC A61M 1/14; A61J 1/05

(57) ABSTRACT

The disclosure relates to a system configured to carry out an extracorporeal blood treatment to a patient which may comprise a sliding cover.

### TECHNICAL FIELD

The present disclosures relate to a system or method for extracorporeal treatment of blood. For example, the present disclosure relates to an extracorporeal blood treatment apparatus conducting a dialysis treatment for example hemodialysis treatment, for example at home.

### BACKGROUND

Extracorporeal blood treatment is typically used to extract undesirable matter or molecules from the patient's blood and/or add desirable matter or molecules to the blood. Such treatment is used with patients unable to effectively remove matter from their blood, such as when a patient has suffered temporary or permanent kidney failure.

This treatment is typically accomplished by removing the blood from the patient, introducing the blood into a filtration unit (for example a dialyzer) where the blood is allowed to flow past a semipermeable membrane. The semipermeable membrane selectively allows matter in the blood to cross the membrane from a primary chamber into a secondary chamber and also selectively allows matter in the secondary chamber to cross the membrane into the blood in the primary chamber, depending on the type of treatment (ultrafiltration (UF) treatment, hemofiltration (HF) treatment, hemodialysis (HD) treatment, hemodiafiltration (HDF) treatment, . . . ).

Currently, the most widely used method of kidney dialysis for treatment of end stage renal disease is hemodialysis. In hemodialysis, the patient's blood is cleansed by passing it through the primary chamber and a dialysate solution through the secondary chamber. During dialysis, arterial and venous parts of blood line convey the patient blood to and from the filtration means (for example a dialyzer). Impurities and toxins are removed from the patient's blood by diffusion or convection across a membrane in the filtration means. Hemodialysis is commonly required three times a week with each dialysis requiring up to four to five hours in a dialysis center or at home where treatment may be more frequent (up to daily) and shorter (down to two hours). During the treatment, the patient is connected to a hemodialysis machine and the patient's blood is pumped through the machine. Catheters are inserted into the patient's veins and arteries so that blood can flow to and from the hemodialysis machine. A large amount of a dialysis solution, for example about 120 liters, is consumed to dialyze the blood during a single hemodialysis therapy.

The dialysis treatment is widely carried up in medical centers, where caregivers operate the dialysis systems and ensure a safe treatment. But more and more treatments are performing at home and the patient is not always accompanied by a caregiver. Thus, it is essential to simplify or to facilitate handling of the dialysis system in order to limit the risks of wrong preparation or actions or the risk of contaminations of sterile elements from the system, and to have a dialysis system which is more compact, more secure and easier to use in order to be used at home while enabling transportation.

### SUMMARY

The disclosures relate to a system or features of a system configured to carry out an extracorporeal blood treatment to a patient. The present document discloses several embodiments of and/or feature of such a system.

A first disclosure relates to a medical system configured to carry out a treatment to a patient. The medical system comprises at least one of an apparatus comprising a housing, and a sliding cover configured to slide over the housing between a first position and a second position.

The sliding cover comprises at least one of a door and walls configured to cover at least a part of the housing in both positions. The sliding cover is configured to define a cavity having variable volume depending on the relative position of the cover to the housing. And the door is configured to allow access to the cavity without removing the cover from the housing.

The door may be movable between an open position allowing access to the cavity and a closed position not allowing access to the cavity. When the sliding cover is in the first position, the door may be movable between an open position and a close position such that the sliding cover may define an at least partially enclosed volume in which a solution bag is protected against external disturbances and/or its heat loss is limited. In one possible embodiment, the system may comprise a securing device configured to secure at leas a part of the solution into the cavity such that the solution cannot contact the inner wall of the sliding cover (as disclosed below).

The medical system may further comprise a guiding means and a sliding means configured to allow slide motion of the sliding cover along the housing. The guiding means and the sliding means may be configured to allow movement along a single axis and to prevent at least one of yawing, pitching, and rolling. The medical system may further comprise a mechanical stop configured to limit the linear motion of the sliding cover along the housing.

The door may comprise a sliding door or a lifting door.

The sliding cover may further comprise an opening configured for the passage of tubes.

The medical system may comprise a lock device so as to maintain the sliding cover in the first or second position. The medical system may further comprise a button configured to unlock the lock device allowing the sliding cover to slide along the housing or to move the sliding cover from the first position to the second position.

The medical system may comprise a tablet support configured to removably secure a tablet on the system. Such a tablet may be configured to provide the user with a user interface. The tablet support may comprise a connector configured to provide the tablet with an electric or electronic connection. For example, the connector may be configured to transfer data from the system to the tablet and/or form the tablet to the system and/or to power the tablet from a power supply arranged into the system. The tablet support may be arranged on the sliding cover.

A second disclosure relates to a system comprising:
a container comprising:
   flexible walls defining a variable volume,
   an inlet tube intended to feed the container with a liquid solution and/or gas, and
   an outlet tube intended to allow the liquid solution to flow out of the container,
a weighing device comprising a tray configured to support the container thereon during use, and
a securing device coupled to the tray which may be configured to secure the container to the tray and/or to control/limit/prevent deformation of at least one of flexible walls such that the container (e.g., the deformation of the container) cannot induce a disturbance in the weighing of the container.

The outlet tube may be configured to extend through the inner compartment of the container. The container may further comprise a lower surface on which at least a part of the outlet tube is secured. For example, an end of the outlet tube may be secured on the lower surface of the container.

The length of the outlet tube extending through the inner compartment may be less than one third of the length of the inner compartment of the container. The outlet tube may be configured to extend further into the inner compartment than the inlet tube.

The container may further comprise a vent device which may comprise a hydrophobic material (such as but not limited to a hydrophobic membrane) configured to allow the passage of gas but not liquid. The vent device may be arranged on a top wall of the container such that the air present in the inner compartment of the container is natively in contact with the vent device. The vent device may be arranged at the middle of the top wall.

The securing device may comprise at least one of a hook, a strap, a flange, and a magnetic attachment.

The system may further comprise a cavity having a lower inner surface and an upper inner surface opposite to the lower inner surface. The container may be configured to be placed inside the cavity between the lower inner surface and the upper inner surface in use. And the securing device may be configured to prevent any contact of the container to the upper inner surface.

The securing device may be configured to secure at least two opposite ends of the container to the tray. In this case, the tray may comprise a first end and a second end opposite the first end. The first end and the second end may comprise a securing device or a part of the securing device. The container may also comprise a first end and a second end opposite the first end. The first and/or the second end of the container may comprise a securing de vice or a part of the securing device (for example hole or other device which may be operatively coupled to the tray (e.g., to the securing device of tray)). The flexible sheets of the container may be welded together and may comprise the securing device or the part of the securing device.

A third disclosure relates to a medical system configured to carry out a dialysis treatment to a patient. The medical system comprises:
A disposable set comprising:
   A cartridge, and
   A first fluid pathway having at least one of a pressure pod and a first tubing line having a first part and a second part,
A reusable machine comprising a recess configured to removably receive the cartridge in use and a pressure transducer configured to be operatively coupled with the pressure pod in use,

Preferentially, the cartridge may comprise a frame, a pod support configured to secure the pressure pod to the frame and a set of alignment devices configured to maintain the pressure pod aligned with the pressure transducer when the cartridge is arranged into the recess while the second part of the first tubing line can move freely without disturbing the pressure measurement.

The cartridge may further comprise a cavity in which U-shaped section of the first tubing line is arranged. The cartridge may further comprise a roller assembly which may be arranged into the cavity.

The frame may comprise a first end against which the U-shaped section of the first tubing line is arranged and a second end, opposite the first end, where the first tubing line exists from the frame.

The reusable machine may further comprise a loading device configured to move the pressure transducer against or away from the pressure pod when the cartridge is received in the recess and/or when the pressure pod is aligned with the pressure transducer.

The recess may comprise a generally planar tray. The cartridge and the recess may be configured to allow the cartridge to slide over the generally planar tray when the cartridge is inserted into or removed from the recess. The generally planar tray may comprise guiding device to facilitate the insertion of the cartridge and/or to guide the cartridge inside the recess.

The frame may comprise a first side having a generally closed shell. The generally closed shell may be configured to slide over and/or to be compressed against the generally planar tray in use. The generally closed shell may extend from the first end to the second end. The pod support may be arranged on an inner surface of the generally closed shell.

The frame may further comprise a second side opposite the first side and having a generally open surface so that the first part of the first tubing line and/or the pressure pod are disposed within the frame but reachable from the outside.

The frame may further comprise a handle configured to be grasped by a user and to protrude from the reusable machine when the cartridge is arranged into the recess. The handle may comprise a set of alignment devices so that a tubing line is arranged in the handle.

The disposable set may further comprise a second fluid pathway comprising a sensor and a second tubing line maintained into the frame by the set of alignment devices. The sensor may comprise at least one of a blood sensor, an air sensor, a pressure sensor, an ammoniac sensor, and a temperature sensor. The reusable machine may further comprise at least one actuator which may comprise at least one of a pump, a valve and a pinch valve.

A fourth disclosure relates to a disposable set comprising:
A cartridge,
A first fluid pathway having at least one of a first pressure pod and a first tubing line having a first U-shaped section,
A second fluid pathway having at least one of a second pressure pod and a first tubing line having a second U-shaped section, and
A third fluid pathway having at least one of a third pressure pod and a first tubing line having a third U-shaped section.

The cartridge may comprise a frame in which the first tubing line, the second tubing line, and the third tubing line are partially arranged. The frame may comprise a first end against which the first U-shaped section, the second U-shaped section, and the third U-shaped section are arranged and a second end, opposite the first end, where the first tubing line, the second tubing line, and the third tubing line enter and exit the frame.

A fifth disclosure relates to a pressure measuring system comprising:
A pressure pod comprising a body with a cavity and an opening, a flexible membrane, an inlet, and an outlet, and
An apparatus comprising:
   A pressure transducer having a sensing portion configured to be removably coupled to the pressure pod,
   A fixed part configured to receive the pressure pod (the fixed part may be configured to be securely fixed/arranged in the apparatus), and
   A movable part, opposite the fixed part (and configured to move relative to the fixed part and/or the pressure pod when the pressure pod is arranged on the fixed part), on which the pressure transducer is arranged,

The pressure pod may further comprise a fluid chamber defined by the cavity and the flexible membrane which closes the opening. The movable part may be configured to (e.g., linearly) move the pressure transducer towards the pressure pod in such a way that the sensing portion comes into contact with the flexible membrane and exerts a force against the flexible membrane to measure a counterforce applied to the sensing portion by the flexible membrane and/or by the fluid within the cavity.

The pressure pod may further comprise a counter-piece configured to maintain the flexible membrane secured on/to the body. The movable part may comprise a surface configured to be in contact with the counter-piece while the sensing portion exerts a force against the flexible membrane.

The flexible membrane may further comprise a protrusion that extends away from the counter-piece. The counter-piece and the surface of the movable part may be configured so that the force is controlled and/or to insure the tightness between the flexible membrane and the body.

The protrusion and the movable part may be configured in such a way that the protrusion makes contact with the sensing portion before the movable part makes contact with the counter-piece.

The pressure pod may further comprise tight features to insure the tightness between the body and the flexible membrane.

The pressure transducer may comprise a load cell.

The system may further comprise a cartridge in which the pressure pod is arranged. The apparatus may comprise a recess in which the cartridge is inserted.

A sixth disclosure relates to a medical system comprising:
A cartridge having a pressure sensor comprising a body with a cavity and an opening, a flexible membrane, at least one port, and a securing device configured to secure the flexible membrane to the body, and
An apparatus comprising:
   A pressure transducer having a sensing portion configured to be removably coupled to the pressure sensor, and
   A movable support on which the pressure transducer is secured and movable along an axis.

The pressure sensor may further comprise a fluid chamber defined by the cavity and the flexible membrane which closes the opening. When the flexible membrane is aligned with the axis, the pressure transducer may be moved toward the pressure sensor until the securing device makes contact with the movable support.

The sensing portion and the flexible membrane may be configured such that when the movable support and the securing device are in contact, the sensing portion exerts a force against the flexible membrane and measures a counterforce applied to the sensing portion by the flexible membrane and/or by the fluid within the cavity.

The flexible membrane may further comprise a protrusion that extends away from the counter-piece. The flexible membrane and the movable support may be configured in such a way that the flexible membrane makes contact with the sensing portion before the movable support makes contact with the securing device.

A seventh disclosure relates to a priming process for a dialysis system comprising an initial solution supply bag storing bicarbonate solution, an additive solution supply bag storing electrolyte, and a dialysate loop circuit having a buffer bag, a dialyzer and a sorbent device.

The priming process may comprise the following steps of:
Filling the buffer bag with a volume of the initial solution,
Priming dialysate loop circuit with the solution stored in the weighing bag,
Adding additive solution to the buffer bag (for example at least partly simultaneously to the priming step to get a mixing solution), and
Passing (e.g., once the priming process is complete or after the priming step) the mixing solution through the sorbent at least until the sorbent has been run-in such that the sorbent can clean the spend dialysate during the treatment (for example: so that the sorbent can operate efficiently from the start of treatment or optimally or efficiently cleans the spent dialysate from the start of treatment).

For further details with regard to the sorbent device, reference may be made to European patent application EP 22205027.0 filed on Nov. 2, 2022, the contents of which are incorporated by reference in the present account.

During the passing step, additive solution may be added to the buffer bag until the mixing solution in the buffer bag becomes a dialysate solution required for dialysis treatment.

As the sorbent can degrade the elements included in the additive solution, during the priming step and/or the passing step, the volume of additive solution added may depend on the operation of the sorbent, or the flow rate of additive solution may be relatively higher than during the treatment, the flow rate of the mixing solution and the flow rate of the additive solution may be configured such that the concentration of elements (e.g., electrolytes such as potassium, calcium, magnesium) of the additive solution increases until the required treatment concentration is reached.

The priming process may further comprise the step of stopping the priming step and/or the passing step once the sorbent has been run-in and/or the solution stored in the buffer bag has become a dialysate solution required for dialysis treatment.

The dialysis system may further comprise a pumping assembly configured to move the solution from the buffer bag, through the dialyzer, afterward through the sorbent device and then back to the buffer bag.

The gas initially stored in the dialysate loop circuit (e.g., pushed by the priming step) or generated by the sorbent may be moved to the buffer bag. The buffer bag may comprise a vent device configured to expel the gas.

Throughout the priming step, the additive solution may be added to the buffer bag. During the priming step, the additive solution may be added continuously to the buffer bag. The additive solution may comprise an element and/or compound selected from the group consisting of water, electrolyte, potassium, magnesium, calcium and combination thereof. Preferentially, the additive solution does not comprise bicarbonate.

The initial solution may comprise an element and/or compound selected from the group consisting of bicarbonate, water, salt, sodium and combinations thereof.

The dialysis system may further comprise a scale configured to weigh the buffer bag.

A eighth disclosure relates to a dialysis system comprising:
a dialysate loop circuit having a buffer bag, a dialyzer and a sorbent device,
a pumping assembly configured to move a solution from the buffer bag, through the dialyzer and the sorbent device and then back to buffer bag,
an initial solution supply bag in fluid communication with the dialysate loop circuit, and
an additive solution supply bag in fluid communication with the dialysate loop circuit, and

The dialysis system may be configured to:
Fill the buffer bag with a volume of the initial solution (optionally),
Prime dialysate loop circuit with the solution stored in the buffer bag,
Add additive solution to the buffer bag (for example, at least partly simultaneously to the priming step to get a mixing solution), and
Pass (e.g., once the priming process is complete or after the priming step) the mixing solution through the sorbent until the mixing solution in the buffer bag becomes a dialysate solution required for dialysis treatment and/or the sorbent device has been run-in.

During the passing step, additive solution may be added to the buffer bag.

The buffer bag may comprise a vent device configured to expel the gas initially stored in the dialysate circuit and moved to the buffer bag and/or the gas generated by the sorbent device.

The dialysis system may be configured to add the additive solution to the buffer bag throughout the priming step. The dialysis system may be configured to add continuously the additive solution to the buffer bag during the priming step.

The additive solution bag may comprise an element and/or compound selected from the group consisting of water, electrolyte, potassium, magnesium, calcium and combination thereof.

The dialysis system may further comprise a scale configured to weigh the buffer bag.

The initial solution bag may comprise an element and/or compound selected from the group consisting of bicarbonate, water, salt, sodium and combinations thereof. Preferentially, the additive solution bag does not comprise bicarbonate.

The pumping assembly may be configured to pump in reverse mode to fill the buffer bag with the initial solution.

A nineth disclosure relates to a dialysis system comprising:
a dialysate loop circuit having a buffer bag, a dialyzer and a sorbent device,
an initial solution supply bag in fluid communication with the dialysate loop circuit,
a first pumping assembly configured to move a solution from the buffer bag, through the dialyzer and the sorbent device and then back to buffer bag and/or to move the initial solution from the initial solution supply bag to the buffer bag,
an additive solution supply bag in fluid communication with the dialysate loop circuit,
a second pumping assembly configured to move the additive solution to the buffer bag, and
an electronic controller configured to control the first pumping assembly and/or the second pumping assembly to:
   Fill the buffer bag with a volume of the initial solution,
   Prime dialysate loop circuit with the solution stored in the weighing bag,
   Add additive solution to the buffer bag, and
   Pass the mixing solution (e.g., once the priming process is complete) through the sorbent until the mixing solution in the buffer bag becomes a dialysate solution required for dialysis treatment and/or the sorbent device has been run-in.

The electronic controller may be configured to control the first pumping assembly and/or the second pumping assembly to add the additive solution to the buffer bag during at least a part of the priming step and/or at least a part of the passing step.

The buffer bag may comprise a vent device configured to expel the gas initially stored in the dialysate circuit and move to the buffer bag and/or the gas generated by the sorbent device.

The electronic controller may be configured to control the first pumping assembly and/or the second pumping assembly to add the additive solution to the buffer bag throughout the priming step.

The electronic controller may be configured to control the first pumping assembly and/or the second pumping assembly to add continuously the additive solution to the buffer bag during the priming step.

The additive solution supply bag may store electrolyte. The additive solution bag may comprise an element and/or compound selected from the group consisting of water, electrolyte, potassium, magnesium, calcium and combination thereof.

The dialysis system may further comprise a scale configured to weigh the buffer bag.

The initial solution bag may comprise an element and/or compound selected from the group consisting of bicarbonate, water, salt, sodium and combinations thereof. Preferentially, the additive solution does not comprise bicarbonate.

The first pumping assembly may be configured to pump in reverse mode to fill the buffer bag with the initial solution.

A tenth disclosure relates to a draining process for a dialysis system comprising a supply bag, a pumping device and a dialysate loop circuit having a buffer bag, a dialyzer, and a sorbent device, wherein the draining process comprises the following steps:
Running the pumping device to fill the buffer bag with a volume of the solution stored in the supply bag (for example before starting the treatment),
Running the pumping device to fill the supply bag with a volume of the solution stored in the buffer bag so as to limit the volume of the buffer bag at the end of the treatment.

The step of filling the supply bag may be performed during the patient's treatment. The step of filling the supply bag may be performed at the end of the patient's treatment. The step of filling the supply bag may be performed after the end of the patient's treatment.

The supply bag may store an initial solution which may comprise an element and/or compound selected from the group consisting of bicarbonate, water, salt, sodium and combinations thereof. Preferentially, the initial solution does not comprise a dialysate solution ready to use.

The supply bag may store an additive solution which may comprise an element and/or compound selected from the group consisting of water, electrolyte, potassium, magnesium, calcium and combination thereof.

The buffer bag may be filled by operating the pumping device in reverse mode. And the supply bag may be filled by operating the pumping device in normal mode.

In another embodiment, the buffer bag may be filled by operating the pumping device in normal mode. And the supply bag may be filled by operating the pumping device in reverse mode.

An eleventh disclosure relates to a dialysis system using a solution for the treatment, the dialysis system comprises:
A loop circuit having:
   A buffer bag,
   A dialyzer, and
   A sorbent,
A first pump,
A second pump,
An initial supply bag in fluid communication with the loop circuit, and
A controller device configured to control the first pump and the second pump according to
   a first mode to move the solution through the loop circuit and,
   a second mode to move the solution from the buffer bag to the initial supply bag.

The first mode may operate the first pump and the second pump. The second mode may operate the first pump and may not operate the second pump. The second pump may be configured to occlude the loop circuit when the second pump is not operating.

The dialysis system may further comprise a fluid connection allowing the initial supply bag to be in fluid connection with loop circuit. The fluid connection may be fluidically arranged between the first pump and the second pump.

The controller device may be further configured to control the first pump and the second pump according to a third mode to move the solution from the initial supply bag to the buffer bag.

The third mode may operate the first pump and does not operate the second pump. The third mode may operate the first pump in a reverse mode while the first mode or the second mode may operate the first pump in a normal operating opposite to the reverse mode.

The dialysis system may further comprise a clamp intended to occlude the line between the dialyzer and the fluid connection, wherein the dialyzer is fluidically arranged between the fluid connection and the second pump.

A twelfth disclosure relates to a dialysis system comprising:
A loop circuit having:
   A buffer bag storing a dialysate solution, A dialyzer, and
   A sorbent,
A first pump,
An additive pump,
An additive supply bag storing an additive solution and in fluid communication with the loop circuit, and
A controller device configured to control the first pump and the additive pump according to
   a first mode to move the solution through the loop circuit and to add additive solution to the loop circuit,
   a second mode to move the dialysate solution from the buffer bag to the additive supply bag.

The present application claims the benefit of the priority of EP22205023.9, EP22205027.0, EP22205030.4, EP22205036.1, EP22205040.3, and EP22205043.7 filed on Nov. 2, 2022 in the name of Nextkidney, the entire disclosure of which is incorporated herein by reference.

### BRIEF DESCRIPTION OF DRAWINGS

The present disclosure will be better understood at the light of the following detailed description which contains non-limiting examples illustrated by the following figures.

FIG. 1 illustrates a potential architecture of the system.

FIG. 2 illustrates a potential fluid circuit of the system.

FIG. 3 shows an exploded view of the machine.

FIG. 4 shows an embodiment of the machine.

FIG. 5 shows an embodiment of the machine.

FIG. 6 illustrates a cross sectional view of an embodiment.

FIGS. 7a and 7b disclose an embodiment with a cover in two positions.

FIG. 8 discloses an embodiment of the cover.

FIG. 9 discloses an embodiment of guiding means.

FIGS. 10a, 10b, and 10c disclose an embodiment in different positions.

FIG. 11 discloses an embodiment of the cover.

FIG. 12 discloses an embodiment with a tablet.

FIG. 13 illustrates a cross sectional view of an embodiment with connecting means for the tablet.

FIGS. 14a and 14b discloses an embodiment with a foldable sensor in two positions.

FIG. 15 illustrates an embodiment of a transfer process.

FIG. 16 illustrates an embodiment of a priming process.

FIG. 17 discloses an embodiment of a bag.

FIGS. 18a and 18b illustrate a cross sectional view of an embodiment of a bag during the treatment.

FIGS. 19a, 19b, 19c, and 19d disclose different embodiments of a bag with attachment means.

FIGS. 20a, 20b, 20c, 20d, and 20e disclose different embodiments of attachment means.

FIGS. 21a and 21b illustrate a cross sectional view of an embodiment of a bag during the treatment.

FIG. 22 discloses an embodiment of a bag.

FIG. 23 illustrates a cross sectional view of an embodiment of a bag during the treatment.

FIG. 24 discloses an embodiment of a bag.

FIG. 25 discloses an embodiment of a bag.

FIGS. 26a and 26b disclose an embodiment of a bag folded.

FIG. 27 shows an exploded view of a dialysate cartridge.

FIGS. 28A and 28B disclose a cross sectional view and an exploded view of a pressure pod.

FIGS. 29A and 29B disclose a top view and an exploded view of the membrane.

FIGS. 30A and 30B disclose different views of the flexible membrane

FIGS. 31A and 31B disclose the pressure pod with the pressure transducer

FIG. 32 illustrates an embodiment of a blood priming process.

FIG. 33 illustrates an embodiment of a blood priming process.

FIG. 34 illustrates an embodiment of a transfer process.

FIG. 35 illustrates an embodiment of a dialysate priming process.

FIG. 36 illustrates an embodiment of a dialysate priming process.

FIG. 37 illustrates an embodiment of a blood return process.

FIG. 38 illustrates a draining process according to an embodiment.

FIG. 39 illustrates a draining process according to an embodiment.

FIG. 40 illustrates a draining process according to an embodiment.

FIG. 41 illustrates a draining process according to an embodiment.

### LIST OF ELEMENTS

1 Fluid circuit of the system
2 Blood circuit
3 Dialysate circuit
4 First filter (e.g. dialyzer)
5 Blood cartridge
6 Dialysate cartridge
7 First bag (e.g. saline bag)
8 Second bag (e.g. empty bag)
9 Third bag (e.g. initial solution bag)
10 Fourth bag (e.g. weighing bag)
11 Fifth bag (e.g. additive bag)
12 Second filter (e.g. sorbent device)
13 First pump
14 Second pump
15 Third pump
16 Fourth pump
17 Vavle or clamp
18 First sensor (e.g. pressure sensor)
19 Second sensor (e.g. air sensor)
20 Third sensor (e.g. level sensor)
21 Fourth sensor (e.g. temperature sensor)
22 Fifth sensor (e.g. ammoniac sensor)
23 Air flow device
24 Connector
25 Arterial line
26 Venous line
27 Drip chamber
28 Additional line
29 Dialysate loop line
30 First supply line
31 Second supply line
32 Patient
33 Weighing scale
34 Warmer
35 Draining line
100 System
101 Resuable part
102 Disposable part
103 Sensor
104 Sensing area
105 Actuator (e.g. valve actuator, pumping device, . . . )
106 Actuation area (e.g. valve, pumping head, . . . )
107 User interface device
108 Patient
109 Processor
200 Machine
201 Cover
202 Tray
203 Housing
204 Dialysate part
205 First opening
206 Blood part
207 Second opening
208 Bottom
209 Door
300 System
301 Housing
302 Cover
302a First part of the cover
302b Second part of the cover
303 Tray
304 Opening
305 Bag
306 Bag
307 Cartridge
308 Tube
309 Cavity
310 Base/Table/Ground
311 Hinge
312 Fisrt motion
313 Second motion
314 Guiding means
315 Sliding means
316 Tablet holder
317 Tablet
318 Locking means
319 Button
320 Shaft
321 Connector
322 Power supply
323 Wireless communication
324 Sliding power connector
325 Power rail
326 Foldable sensor
400 Weighing scale
401 Warmer
402 Heating contact
403 Corver
404 Attachment means
405 Liquid
406 Gas
407 First tube
408 Second tube
409 Storage compartment
410 Edge
411 Flexible wall
412 Tube attachment
413 Hydrophobic membrane
414 Opening
415 Attachment means
1200 Frame
1201 Handle
1202 Tube guide
1203 Pod support
1204 Frame of the pump
1205 Flexible tube
1206 Roller
1207 Roller holder
1208 Mechanical stop
1209 Roller assembly
1210 Planar surface
1211 Guiding element
1212 Pressure pod
1220 System
1221 Cartridge or pressure pod
1222 Body
1223 Cavity
1224 Flexible membrane or diaphragm
1225 Securing device
1226 Apparatus
1227 Movable part
1228 Fixed part
1229 Pressure transducer
1230 Sensing portion
1231 Protrusion
1300 Strain sensor
1301 Body
1302 Membrane
1303 Membrane holder
1304 Cavity
1305 inlet/outlet port
1306 Rayon
1307 Inner bend radius
1308 Outer bend radius
1309 Protrusion
1310 Protrusion width
1311 Protrusion thickness
1312 Thickness of edge
1313 Internal thickness
1314 Overall thickness
1315 Inner wall
1316 Groove
1317 Opening

### DETAILED DESCRIPTION

The following detailed description includes references to the accompanying drawings, which form a part of the detailed description. The drawings show, by way of illustration, specific embodiments in which the disclosure may be practiced. These embodiments, which are also referred to herein as “examples,” are described in enough detail to enable those skilled in the art to practice the disclosure. The embodiments may be combined, other embodiments may be utilized, or structural, logical, and electrical changes may be made without departing from the scope of the present disclosure. The following detailed description is, therefore, not to be taken in a limiting sense, and the scope of the present disclosure is defined by the appended claims and their equivalents.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the claims, the singular forms “a”, “an”, and “the” encompass embodiments having plural referents, unless the content clearly dictates otherwise.

As used in this specification and the claims, any direction referred to herein, such as “top”, “bottom”, “left”, “right”, “upper”, “lower”, and other directions or orientations are described herein for clarity in reference to the figures and are not intended to be limiting of an actual device or system unless the content clearly dictates otherwise. Devices and systems described herein may be used in several directions and orientations.

As used in this specification and the claims, “have”, “having”, “include”, “including”, “comprise”, “comprising” or the like are used in their open-ended sense, and generally mean “including, but not limited to”.

As used in this specification and the claims, the term “or” is generally employed in its sense including “and/or” unless the content clearly dictates otherwise.

As used in this specification and the claims, “at least one of A, B, and C”, “at least one of A, B or C”, “selected from the group consisting of A, B, C, and combinations thereof” or the like are used in their open ended sense including “only A, or only B, or only C, or any combination of A, B and C” unless the content clearly dictates otherwise.

### Reusable and Disposable Parts

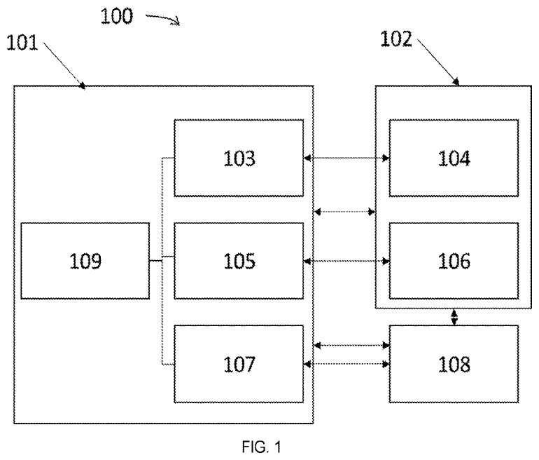

According to one embodiment of the disclosure as shown by FIG. 1, the system (100) may comprise a reusable part (101) and a disposable part (102). The disposable part (102) may comprise the elements which have to be discarded after a predetermined number of uses, for example, after a single treatment. The working life of the disposable part (102) may directly depend on the number of treatments. These elements may be the elements which have been wetted by a fluid such as the medical fluid (such as dialysate) or by the patient fluid (such as blood).

The reusable part (101) may comprise the expensive elements for example the sensor (103), the electronic part, the user interface device (107), the actuator (105) of the valve or of the pump, the processor (109) or the memory. The reusable part (101) may be configured to be successively used with several disposable part (102). The reusable part (101) may comprise components which may be replaced when the components are too worn, become broken or after a predetermined period of time, but much longer than a single treatment. Changing the reusable part may depend on the wear of the components.

The Reusable part (101) may be configured to be operatively coupled to the disposable part (102). A sensor (103) may be configured to be operatively coupled to a sensing area (104) of the disposable part. An actuator (105) may be configured to be operatively coupled to an actuation area (106) of the disposable part (102). The reusable part (101) (e.g. the user interface device (107) such as, but not limited to, a graphical user interface (GUI)) may be configured to provide information to the patient (108) and/or to receive instruction from the patient (108). At least one of the sensor (103), the actuator (105) and the user interface device (107) may be connected to an processor (109). The disposable part (102) may be connected to or in contact with the patient (108) at least during the treatment.

According to one possible embodiment, the system may be configured to carry out a dialysis treatment at home (such as, but not limited to, a hemodialysis treatment or an extracorporeal treatment). In order to limit the volume of dialysate required for the treatment, the system may be configured to recirculate the dialysate solution over the treatment. In this case, once the dialysate solution is deemed spent (e.g., it has passed through the dialyzer), the dialysate solution must be cleaned by a sorbent device and electrolytes must be added to the cleaned dialysate before it is reused (e.g., passing through the dialyzer again).

In some possible embodiment, during a hemodialysis treatment, the ultrafiltrate (liquid removed from the patient by ultrafiltration through the dialyzer), the additive solution (which may comprise electrolytes) and the dialysate are collected in a single bag (also called weighing bag or fourth bag thereafter or buffer bag). At the beginning (e.g., initially or before starting the treatment), the weighing bag may be empty, afterward it may be filled with a first volume of 2 L (which may comprise dialysate solution or a solution for preparing the dialysate or a priming solution) and then, the weighing bag may be filled with a volume up to 5 L of liquid for example over the treatment (due to the ultrafiltrate and the added electrolyte).

### Fluid Circuit

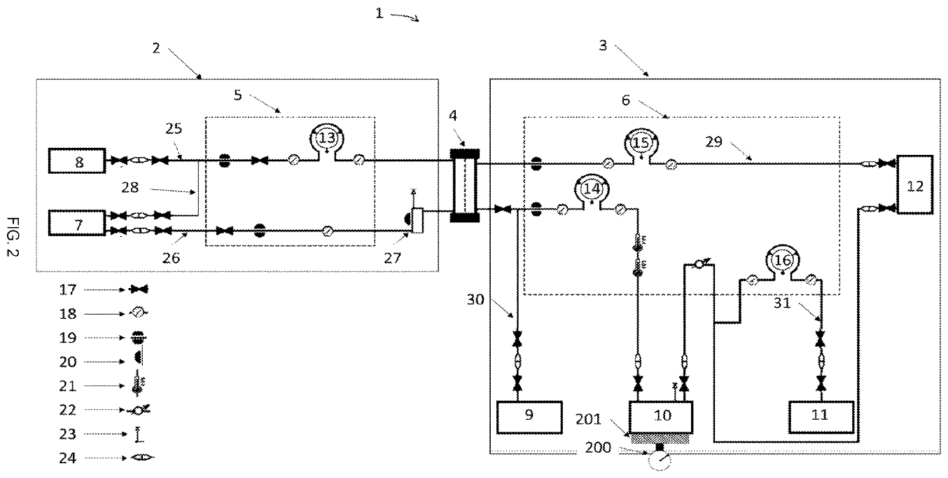

According to a possible embodiment shown by FIG. 2, the system (e.g. as disclosed above) may comprise a fluid circuit (1). The fluid circuit of the system (1) may comprise a blood circuit (2), a dialysate circuit (3), and a first filter (e.g., dialyzer) (4). The elements of the fluid circuit wetted by a fluid may be a part of the disposable part (e.g. as disclosed above). The system may comprise at least one of a first sensor (18) (e.g., pressure sensor), a second sensor (19) (e.g., air sensor), a third sensor (20) (e.g., level sensor), a fourth sensor (21) (e.g., temperature sensor), and a fifth sensor (22) (e.g., ammonia sensor). These sensors may be a part of the reusable part (e.g., as disclosed above) and/or arranged into the reusable part.

The blood circuit (2) may comprise at least one of a connector (24), an arterial line (25), a venous line (26), and a first pump (13). The arterial line (25) and/or the venous line (26) may be intended to be connected to the patient via for example a catheter (not shown here), for example during the treatment. The first pump may be configured to move a fluid (for example blood of patient) from the arterial line to the venous line in a normal operation (for example during the treatment) and from the venous line to the arterial line in a reversed operation (for example during a part of the priming process and/or blood return process). The first pump may be controlled by and/or operatively coupled to the processor.

The blood circuit (2) may further comprise at least one of a first pump (13), a drip chamber (27), a pressure sensor (18), an air sensor (19), a level sensor (20), a valve or clamp (17), an additional line (28), a first bag (7), and a second bag (8). The additional line (28) may be connected to the arterial line (25) and/or to the venous line (26) via for example a T connector. The additional line may comprise at least one of a valve, a clamp, a connector. The first bag (7) may be connected to at least one of the venous line (26), the arterial line (25) and the additional line (28) via for example a connector. The second bag (8) may be connected to at least one of the venous line (26), the arterial line (25) and the additional line (28) via for example a connector. The drip chamber (27) may comprise an air flow device (23). At least some of the elements listed above can be arranged in a cartridge, e.g., a blood cartridge (5). The blood circuit may be connected or operatively coupled to the first filter (4).

The dialysate circuit (3) may comprise at least one of a dialysate line (29), a second pump (14), a third pump (15), a second filter (12) (for example a sorbent device), and a fourth bag (10). The dialysate circuit (3) may further comprise at least one of a first supply line (30) connected to a third bag (9) and a second supply line (31) connected to a fifth bag (11). The dialysate line may define a loop circuit through which the dialysate solution is moved by at least one of the second pump (14) and the third pump (15). The loop circuit of the dialysate line may comprise at least one of the fourth bag (10), the second pump (14), a dialysate compartment of the first filter (4), the third pump (15) and the second filter (12). Once the dialysate solution has passed through the second filter, the dialysate line can be configured so that the dialysate solution reaches the fourth bag (10).

The second supply line may comprise at least one of a fourth pump (16), a valve, a clamp, a connector. The second supply line may be fluidly connected to the dialysate line (29) via a T shape connector, for example downstream of the second filter (12) and/or upstream of the fourth bag.

The third bag (9) and/or the fifth bag (11) may be removably connected to its respective supply line via for example a connector (24). The fourth bag (10) may be removably connected to the dialysate loop line (29) via for example a connector (24). The dialysate circuit may be connected or operatively coupled to the first filter (4).

The dialysate circuit may further comprise at least one of a pressure sensor (18), a temperature sensor (21), an ammonia sensor (22), a connector (24), and a valve or clamp (17). At least some of the elements listed above may be arranged in a dialysate cartridge (6).

The fourth bag (also called weighing bag) (10) may be arranged on a warmer (34) and/or a weighing scale (33). The weighing scale may be connected to a processor which may be configured to determine the volume (or the weight or a data related to the weight or volume) of fluid (liquid) stored in the weighing bag (10).

The third bag (9) may initially store an initial solution. The initial solution may comprise a priming solution, a dialysate solution (ready to use), a solution used to prepare the dialysate solution. The third bag may be configured to store a volume of fluid comprised between 0.5 L and 4 L, preferentially between 1 L and 3 L, for example 2 L.

The blood circuit (2) and the dialysate circuit (3) may be fluidically connected to the first filter (4) for example a dialyzer. The first filter may comprise a blood compartment connected to the blood circuit and a dialysate compartment connected to the dialysate circuit. Both may be separated by a permeable membrane.

### Machine/Apparatus

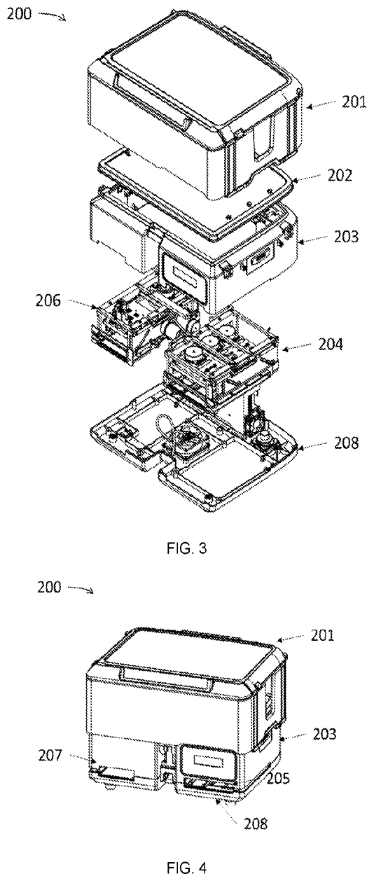

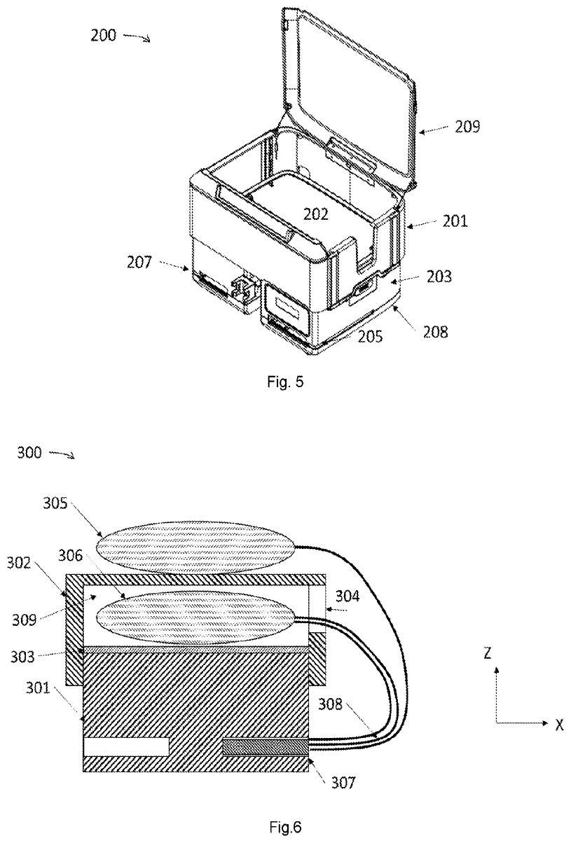

In one possible embodiment as shown by FIGS. 3, 4, and 5, the system may comprise a machine (200) which may be a reusable part. The machine (200) may comprise at least one of a cover (201), a tray (202), a housing (203), a dialysate part (204), a first opening (205), a blood part (206), a second opening (207), and a bottom (208).

The dialysate part (204) and the blood part (206) may be arranged inside the housing (203). The first opening (205) may be configured to enable the insertion of at least a part of a dialysate circuit (e.g., a dialysate cartridge) so that the dialysate part (204) can be operatively coupled to the dialysate circuit in use. The second opening (207) may be configured to enable the insertion of at least a part of a blood circuit (e.g., a blood cartridge) so that the blood part (206) can be operatively coupled to the blood circuit in use. The blood part and/or the dialysate part may comprise at least one of a loading device, sensor, actuator, . . .

The tray (202) may be configured to receive, weigh, and/or heat a solution bag (such as, but not limited to, the fourth bag). The tray (202) may be arranged on/above the housing (203). The tray may be arranged in such a manner that the cover (201) define a cavity in which the solution bag is kept warm and/or protected from external disturbance. The cover (201) may comprise a door (209) so that the tray (202) can be accessed without removing the cover (201) from the housing (203). In one embodiment as described thereafter, the cover may be configured to slide over the housing, providing a variable volume to the cavity.

### Sliding Cover

FIG. 6 illustrates a cross sectional view of a possible embodiment, the system (300) which may comprise a housing (301) and a cover (302). The cover (302) may define a cavity (309) in which a bag (306) (for example a weighing bag) may be stored at least during the treatment. For example, the inner wall of the cover may define the lateral and/or the upper limits of the cavity. The machine may comprise a tray (303) (as disclosed above) configured to weigh and/or heat the bag placed in the cavity (309). The cover may comprise a tray configured to receive and/or to keep/contain a bag (305) (for example a supply bag) on it. At least one of the bags may comprise at least one tube (308) connected to a cartridge (307) which may be operatively coupled to the machine. In case where the bag (306) comprises a tube as disclosed above, the cover may comprise an opening (304) such that the tube can pass through the cover (302). The cover may be configured to at least partially surround the housing.

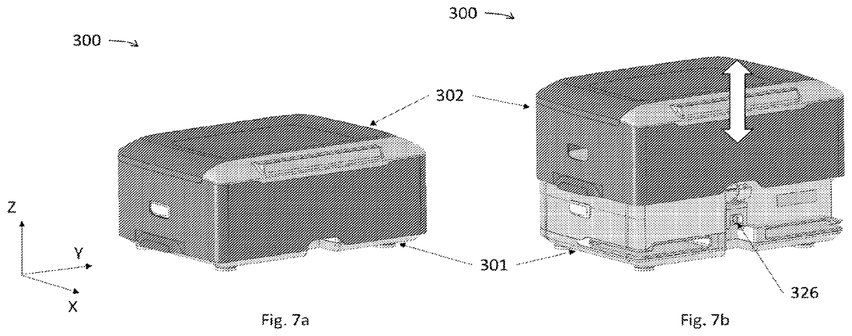

According to one embodiment as disclosed by FIGS. 7a and 7b, the cover (302) may be configured to move relative to the housing (301). For example, the cover (302) may be configured to slide relative to the machine (301) in at least one dimension such as in Z-axis. The cover position illustrated in FIG. 7a may be optimal for transport and/or storage of the machine (e.g., when the machine does not carry out a treatment) while the cover position illustrated in FIG. 7b may be required during the treatment (e.g., when the machine is carrying out a treatment).

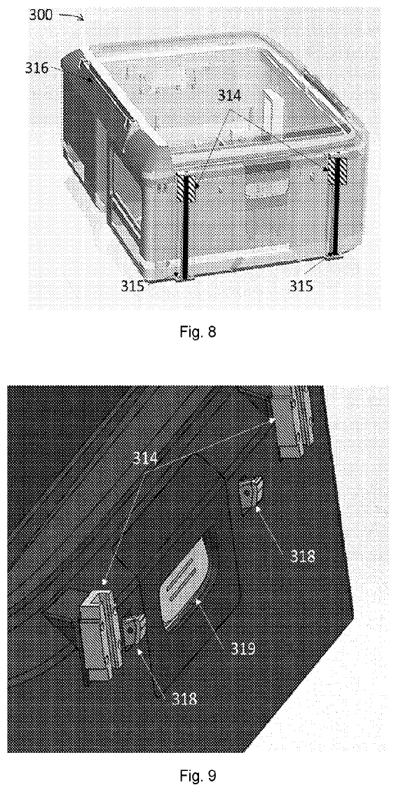

As disclosed by FIG. 8, the system may comprise at least one of a guiding means (314) and a sliding means (315) configured to slide along the guiding means (314). The system may comprise at least two guiding means and at least two sliding means. The sliding and guiding means may be arranged on at least two opposite sides of the system. In one embodiment, the system may comprise two opposite sides and each of these sides may comprise two guiding means and two sliding means. In other terms, the system may comprise four guiding means and four sliding means, whereby two guiding/sliding means may be arranged on two opposite sides of the system.

The guiding means and sliding means may be configured to allow movement along a single axis (e.g., the Z axis) and to prevent at least one of yawing, pitching, and rolling.

The sliding means may be arranged on the cover (for example on the inner wall of the cover) and the guiding means may be arranged on the housing. The sliding means may comprise a T-shaped strip which slide through a T-shaped opening of the guiding means (314) as disclosed by FIG. 9.

The system may further comprise a locking means (318) configured to block the cover in a determined position. The system may further comprise a button (319) to unlock the position of the cover. The locking means may comprise a hook, a finger, or any element able to lock the position of the cover.

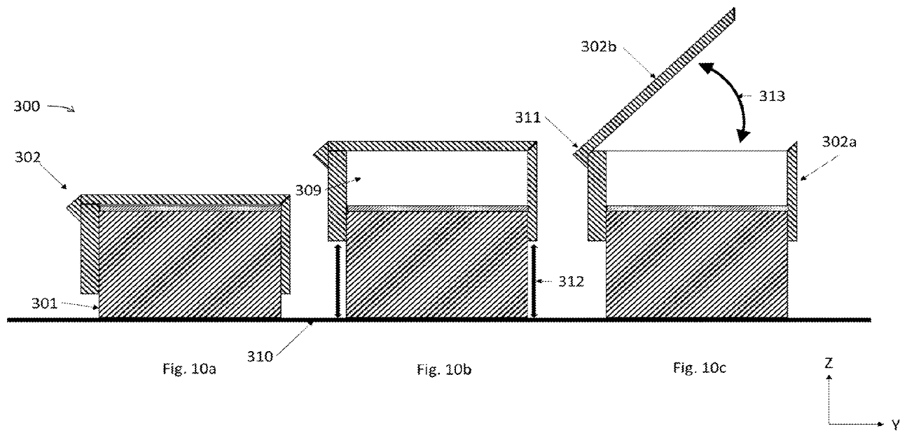

According to one embodiment as disclosed by FIGS. 10a, 10b, and 10c, the cover (302) may comprise at least two parts, a first part (302a) configured to slide over/along/relative to the housing and a second part (302b) configured to provide an access to the cavity (309). The cover may define a variable-volume cavity. As the cover lowered, the volume of the cavity decreases, and as the cover rises, the volume of the cavity increases. The cover (302) may comprise at least one of a first position (which provides a compact size of the system with the smallest volume of the cavity (309)), a second position (required for the treatment with a determined volume of the cavity for example able to contain a full bag), and a third position (required to place or deposit a bag in the cavity and provide an access to the cavity (309)).

The second part may comprise a door. The door may comprise a hinge (311) coupled to the first part of the cover as disclosed in FIG. 11. The hinge may comprise a shaft (320) for joining the first and the second part of the cover. A mechanical stop may limit the motion of the door.

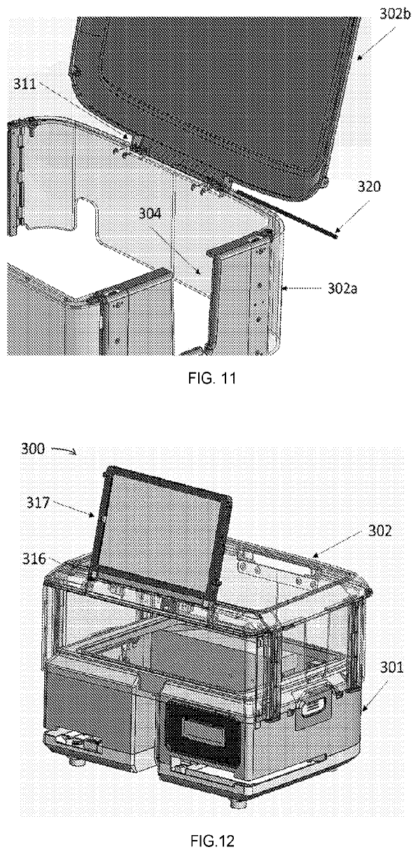

According to one embodiment as disclosed by FIG. 12, the system may further comprise a (remote/removably) tablet (317) which may be used as user interface of the system. The cover (302) may comprise a tablet holder (316) configured to receive and/or keep the tablet on the system.

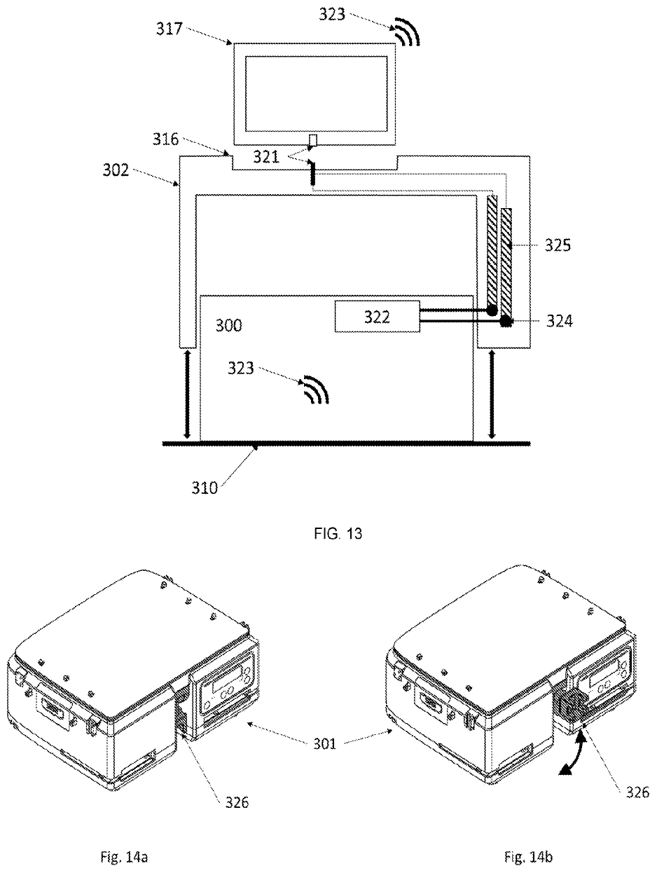

Focusing on FIG. 13, the system may comprise a recharging device configured to recharge the battery of the tablet (317). For example, the tablet holder may comprise a connector (321). The machine may comprise a power supply (322) adapted for the tablet. The system may further comprise a sliding power connector (324) and a power rail (325) configured to be operatively coupled such that the power supply (322) of the machine can provide power to the connector (321) of the tablet holder (or cover). The sliding connector may be configured to slide (and maintain an electric connection) along the power rail (325) when the cover slide along the machine. The tablet and the machine may communicate wirelessly. In another embodiment, the power supply (322) is connected to the connector (321) by flexible wires. The system may further comprise a guide means (such as but not limited to a cable tray) for guiding the flexible wires when the cover is slid over the housing.

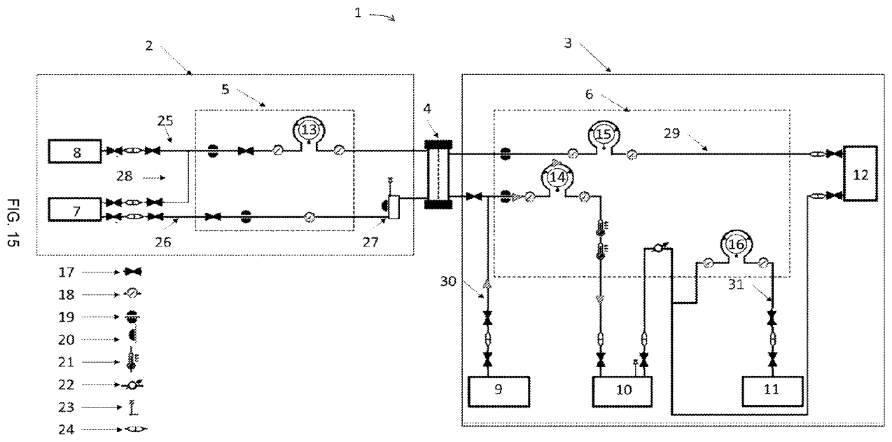

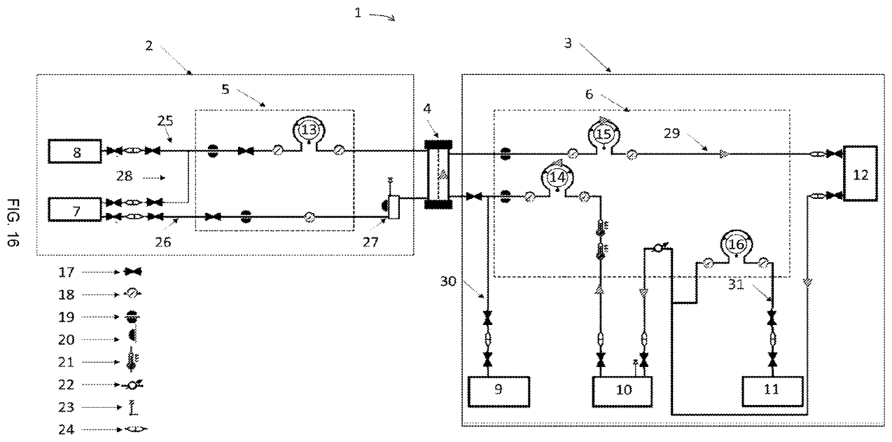

Focusing on FIGS. 14a and 14b, the system may further comprise a foldable sensor (326) configured to have a first position (which may provide a compact size of the system) (see FIG. 14a) and a second position (which may be required during the treatment) (see FIG. 14b). The first position may comprise a folded position and the second position may comprise an unfolded position. The system may comprise a first surface having a recess configured to position the foldable sensor in the first position such that the foldable sensor does not protrude from the first surface, while the foldable sensor may protrude from the first surface in the second position. The second position may allow receipt of an element to be monitored, such as a drip chamber.

### Bag

In one possible embodiment, the system comprises a volume-variable bag (such as but not limited to the fourth bag (10)) configured to increase its volume over the treatment. As described above, the fourth bag (10) may act as a buffer container of the dialysate circuit (more particularly loop of the dialysate line). The fourth bag is configured to contain the dialysate solution to be used for the treatment. The fourth bag is further configured to receive at least one of the sorbent-cleaned dialysate solution, the ultrafiltrate, the gas (such as but not limited to CO2) generated by the chemical reaction in the sorbent and a concentrate solution.

The fourth bag may be initially empty of liquid (for example, the fourth bag may be delivered empty to the user (patient), or the fourth nag may be empty until the start of the treatment or the priming step). In this case, before starting the treatment, the fourth bag must be filled of a dialysate solution or other solution. FIG. 15 illustrates a transfer process for filling the fourth bag (10) with the solution initially stored in the third bag (9). The valve/clamp dedicated may be opened while the others may be closed. The second pump (14) may be operated to move the initial solution from the third bag (9) to the fourth bag (10). The second pump may be operated in a reverse direction during the transfer process while during the treatment the second pump (14) may be operated in a normal direction (e.g., from the fourth bag to the first filter). The reverse direction may be opposite to the normal direction.

This step may be launched by the user or the processor and may be stopped by the processor. The processor may monitor the proper operating of the transfer process by using at least one of a pressure sensor (18), a weighing scale (which weighs the fourth bag (10)) and a sensor monitoring the second pump (14). The processor may stop the transfer process depending on the measurements of at least one of these sensors for example if the measurement reaches a determined threshold or does not reach another determined threshold for a determined period of time. The processor may monitor the weight of the fourth bag (10) during the time period of the transfer process.

The transfer process may be stopped by the processor due to a failure, the end of the process and/or after a determined time period (during which the second pump runs), a number of actuations of the second pump, a number of revolutions of the second pump (if the second pump is a peristaltic pump), if the pressure measurement sharply dips (in case of the third bag (9) being empty), if the weight of the fourth bag reaches a determined threshold. If no failures occur during this process (e.g., for a determined time period), the processor may stop the second pump and/or may determine that the transfer process is completed.

In the case where the third bag (9) is located above the fourth bag (10), the transfer process may be (also or a partly) performed by gravity. The transfer process may be used as part of the dialysate priming process. In one embodiment, the fourth bag (10) is placed on a warmer which may comprise a weighing scale. During or after the end of the transfer process, the warmer may heat the initial solution in the fourth bag (10).

FIG. 16 illustrates a possible dialysate priming process. This process may allow at least to prime the dialysate loop line (29). During this process, the second pump (14) and the third pump (15) are operated to move the solution stored in the fourth bag (10) and push the fluid (e.g. gas) initially stored in dialysate circuit (for example in the line (29), in the dialysate compartment of the first filter (4) and in the second filter) to fourth bag (10).

To prime the dialysate loop line (29), the liquid solution is moved through the lines and reaches successively the second pump, the first filter, the third pump, the second filter and then back into the fourth bag. The processor may be configured to monitor the weighing scale of the fourth bag (10).

The sorbent (12) may comprise at least one of active carbon, ion exchangers (such as zirconium phosphate and/or hydrous zirconium oxide), and one or more enzyme (e.g. urease). During the treatment as explained above, the sorbent may be configured to remove the toxin (such as urea and other) through a chemical reaction with the component(s) comprised in the sorbent. This chemical reaction may generate gas (such as CO2). The sorbent may also remove other element present in the dialysate solution such as electrolyte which may be added downstream the sorbent (12) for example via the second supply line (31).

Therefore, the aim(s) of the weighing bag (fourth bag (10)) may be:
Collection of the liquids from the dialysate loop line (29), for example the liquid coming from regeneration process (the dialysate cleaned by the sorbent device (second filter (12)) and ultra-filtration coming from the patient (due to the first filter (dialyzer (4)),
Collection of the CO2 gas (caused by the chemical reaction in the sorbent) and the gas due from the priming process,
Separation of the gas from the liquid,
Evacuate of the gas present inside the weighing bag (10),
Enable the determination of a data related to the weight of the volume collected inside the weighing bag,
Enable optimized heating (for example a homogenous heating of the liquid and/or preventing energy loss between the warmer plate and the weighing bag) of the liquid solution stored inside the weighing bag,
Enable a volume buffer,
Mixing the additive solution (electrolyte) with the cleaned dialysate solution, and/or
Limit or prevent gas stored in the weighing bag from reaching the first dialyzer (4).

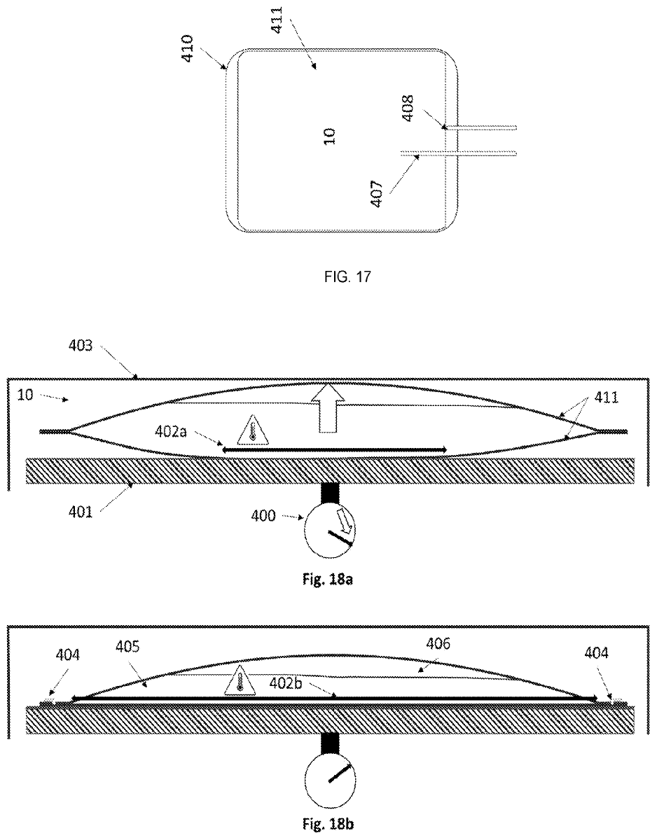

FIG. 17 discloses a potential embodiment of the fourth bag (10). The fourth bag may comprise at least one of a first tube (407), a second tube (408), a storage compartment (409), edge(s) (410), and at least one flexible wall (411). The portion of the first tube that extends into the storage compartment may be longer than the portion of the second tube that extends into the storage compartment. The first tube can be used as a fluid outlet (fluid going to the dialyzer) while the second tube can be used as a fluid inlet (fluid coming from the sorbent device) and vice-versa. The storage compartment may be defined by two flexible walls joined to each other for example at their edges. The first tube (407) and the second tube (408) may be arranged on the same edge (410) if the fourth bag.

The weighing bag may be put horizontally on the warming/weighing plate and a cover (201/403) may be arranged above to define a cavity which may protect the weighing bag and may insulate the cavity.

Several problems may be solved:
Limit the risk of recirculation of the fluid coming from the regeneration to the outlet,
No suck the gas from the weighing bag to the outlet tube port.
Evacuate CO2 gas,
Have a good contact with the warming plate, and/or
Stay inside the dedicated volume defined by the cavity and assure than the bag doesn't touch the cover to do not perturbate the weighing function.

Focusing on FIG. 18 a, the fourth bag (10) is placed on the weighing/warming plate and under a cover (403). Due to the increased volume and flexible walls (411) of the fourth bag (10), the height of the fourth bag may touch the cover (403) and may induce erroneous measurements of the weighing scale (400). Furthermore, due to the flexible walls (411), a small portion of the fourth bag is in contact with the weighing/warming plate which induce a loss of heating efficiency.

In one embodiment as disclosed by FIG. 18 b, the system may comprise attachment means (404) configured to hold a surface of the fourth bag in contact with the warmer plate and limit the height of the fourth bag. When the fourth bag is attached to the warmer plate, the heating contact (402b) is greater than the heating contact (402a) when the fourth bag is not attached to the warmer plate. This may improve the efficiency of the warmer.

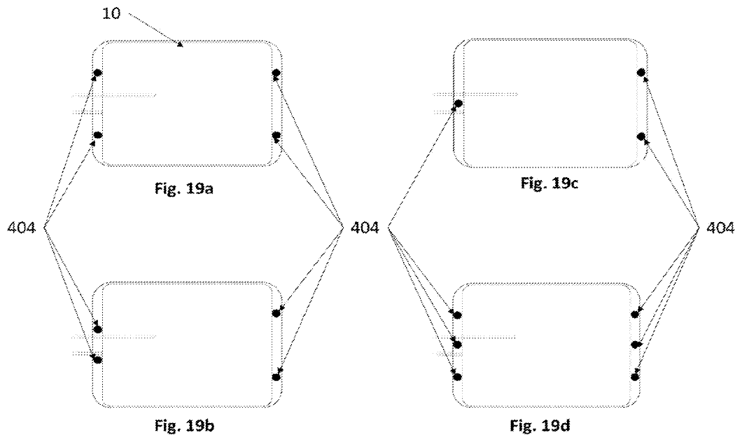

Focusing on FIGS. 19a to 19c, the attachment means may be arranged in different location. In one embodiment, at least one attachment means (404) may be arranged on the edge comprising at least one of the first tube and the second tube. In one embodiment at least one attachment means may be arranged on a first edge and at least one attachment may be arranged on a second edge. The first edge may be located opposite the second edge.

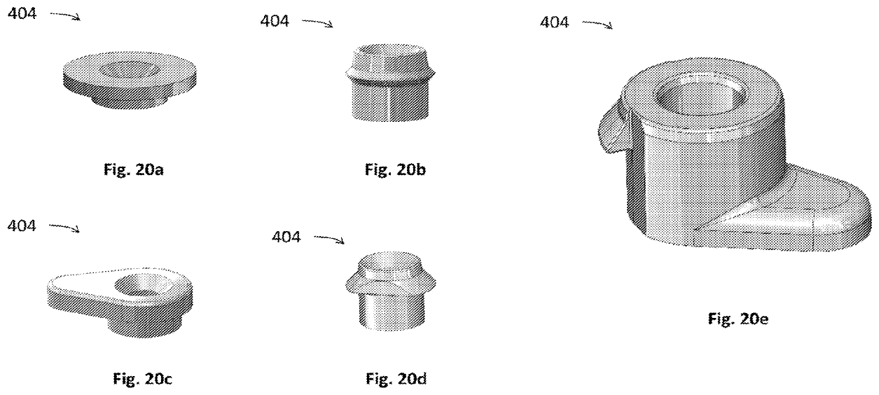

The attachment means may comprise at least one of a hook, a clip, magnetic element, and cooperative elements. FIGS. 20a to 20e disclose several attachments means. These attachment means may be arranged on the weighing/warming plate and the fourth bag may comprise cooperative element (such as through holes arranged to at least one edge of the fourth bag). The attachment means may comprise at least one of an elongated body, a hook, and a reinforced foot opposite the hook.

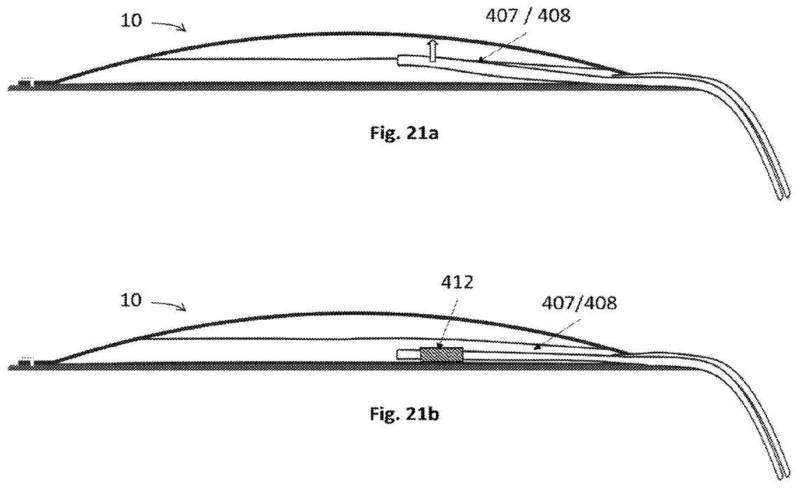

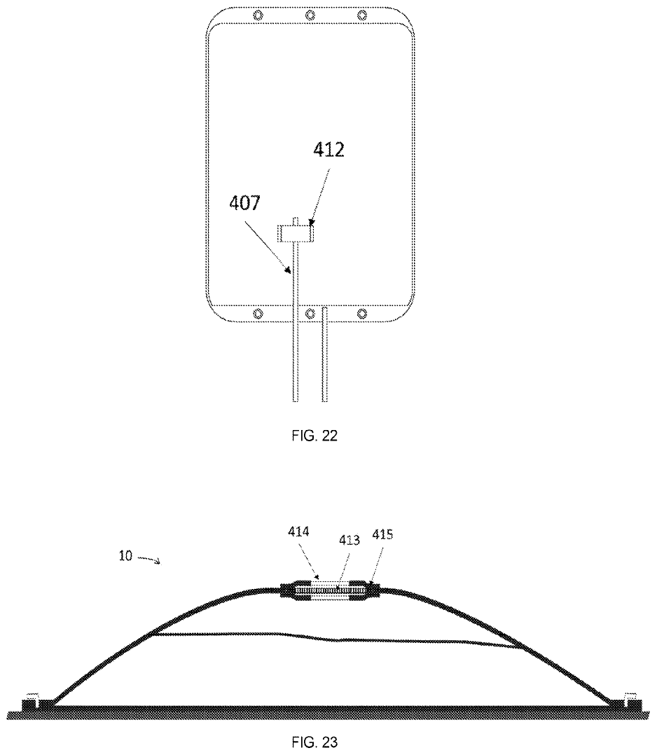

Focusing to FIGS. 21a, 21b, and 22, in order to ensure that the outlet tube cannot provide only liquid, the outlet tube (407 or 408) may be kept submerged in the storage compartment (409). For example, the outlet tube may be attached (412) to the flexible wall intended to be in contact with the weighing/warming plate.

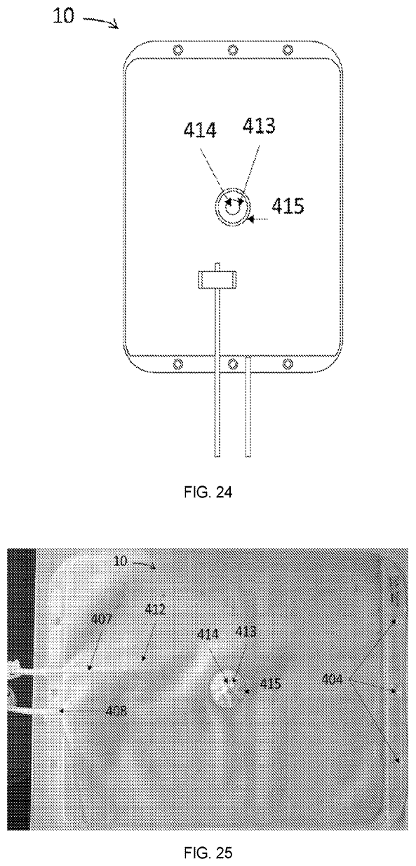

Focusing to FIGS. 23 and 24, in order to enable gas to be expelled from the storage compartment (409) of the fourth bag (10), the fourth bag (10) may comprise at least one of a hydrophobic membrane (414) and an opening (4213) in the top wall. The top wall may comprise a flexible wall (411). The hydrophobic membrane may be rigidly fastened to top wall via an attachment means (415) (for example, weld, glue, . . . ). The size of the hydrophobic membrane may be larger than the size of the opening in order to protect at least a part the membrane.

The hydrophobic membrane may be configured such that any liquid fourth bag cannot pass the hydrophobic membrane (414) and will remain in the storage compartment while gas can pass through the hydrophobic membrane (414). The hydrophobic membrane may comprise a porous material. A pore size of the membrane material can be 0.1-10 μm, in particular 1-7 μm. To ensure that no bacteria enter the storage compartment during treatment, the hydrophobic membrane may be configured to expel gas only when the fluid pressure in the storage compartment is higher than the environment outside the fourth bag. In one embodiment. To ensure that the hydrophobic membrane is in contact with the gas present in the storage compartment, the hydrophobic membrane may be arranged in the middle of the top wall in at least one dimension.

FIG. 25 shows an embodiment of the fourth bag.

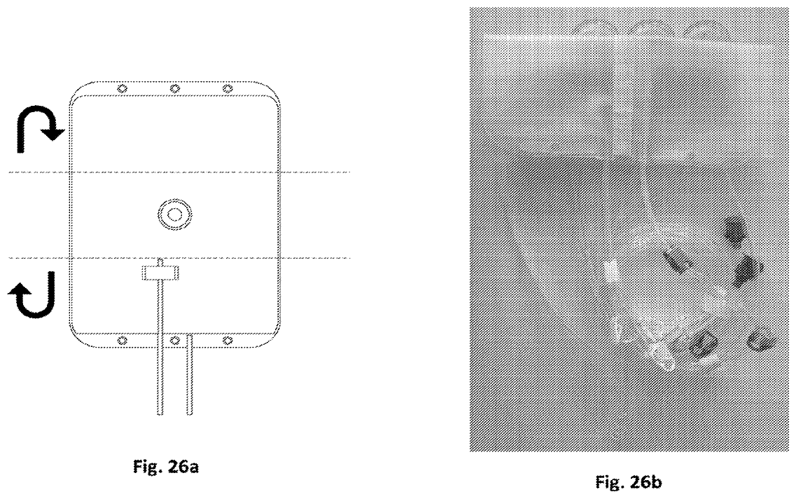

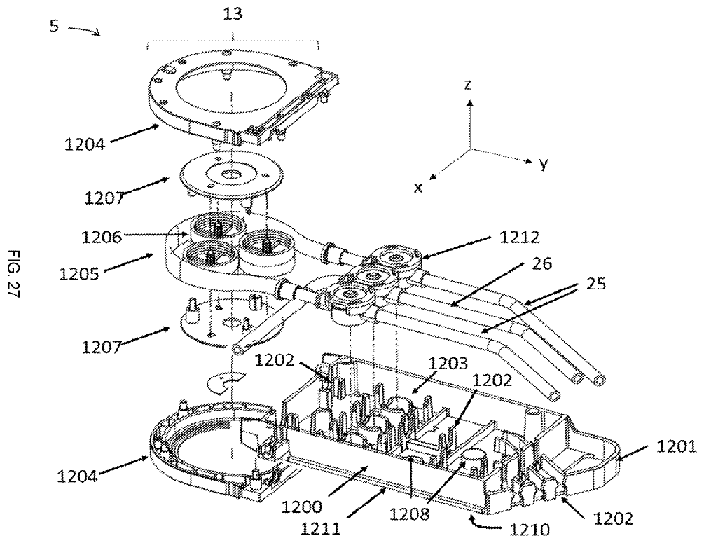

In order to limit the size for storage and transportation of the disposable set. The fourth bag may be foldable in at least 2 portions in at least one dimension. The fold line may depend on at least one of the size and/or the position of the hydrophobic membrane and the length and/or the position of the first tube and/or second tube. In the embodiment shown by FIGS. 26 and 26, the fourth bag is folded in three in the direction of the width.

### Cartridge(s)

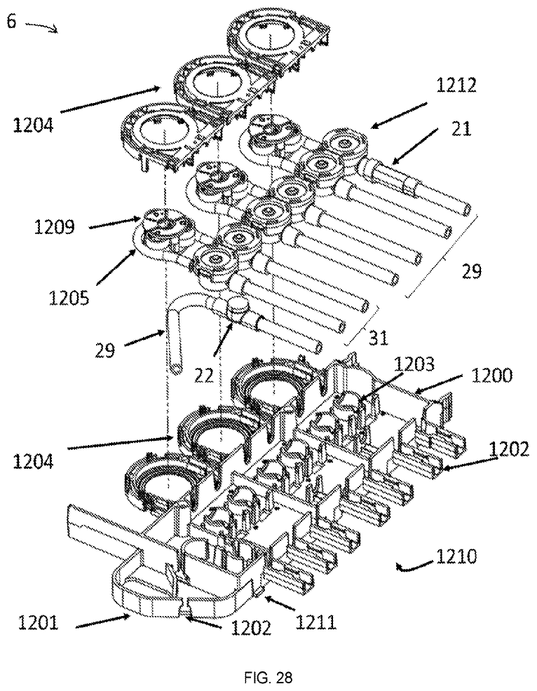

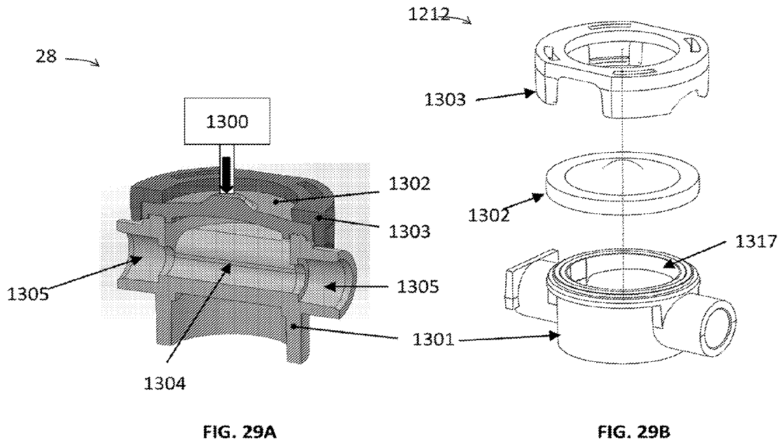

The system may comprise at least one cartridge (for example a blood cartridge (5) and/or a dialysate cartridge (6)). An example of a blood cartridge is shown by FIG. 27. An example of a dialysate cartridge is shown by FIG. 28.

The cartridge may comprise a frame (1200). The frame may comprise a first side and a second side. The first side may comprise a substantially planar surface (1210). The second side may be configured to receive a tube assembly and may comprise at least one of an opening, a support element (such as pod support (1203)), and a tube guide (1202). The frame may further comprise at least one lateral edge in order to protect the tube assembly when it arranged on the second side.

The tube assembly may comprise at least one of a tube, a pressure pod (1212), and a flexible tube (1205) intended to the pump. The second side of the frame may be configured to receive the tube assembly (already assembled) and secure the tube assembly in the frame.

The pod support (1203) may comprise at least one of a seat and a fastening means (such as a clip element, or other fastening element know by the skilled person), either or both of which may be configured to hold the pressure pod (1212) in at least one direction (x, y, or z). For example, the seat may configured to hold the pressure pod (1212) at least in one direction (x, y or z) or in at least two directions (x+y or x+z or y+z) and the fastening means (such as a clip element, or other fastening element know by the skilled person) configured to hold the pressure pod (1212) at least in one direction (x, y or z) or in at least two directions (x+y or x+z or y+z).

The cartridge may further comprise a pump head which may comprise a pump frame (1204), and a roller assembly (1207). The roller assembly may comprise a roller holder (1207) and one or more rollers (1206). The pump frame (1204) may comprise an upper part and a lower part configured to be joined together forming a cavity in which may be arranged the roller assembly (1209) and the flexible tube (1205). A pump actuator (not shown here which may be arranged into the reusable part of the system) may be configured to provide a rotational movement of the roller assembly and may exert a force against the flexible tube in order to compress the flexible tube.

The pump frame (1204) may be fastened to the cartridge frame (1200) to provide a single piece. The pump frame may comprise fastening elements configured to cooperate with the cartridge frame (such as pin or clip or other mechanical fastening element known by the skilled person) in such a way when the upper part and the lower part are coupled to the cartridge frame, the pump frame is rigidly fastened to the cartridge frame. Once the upper part and the lower part joined and coupled to the cartridge frame, the tube assembly may be fastened to the cartridge. With this configuration, the cartridge and the tube assembly may be easily assembled.

The frame may further comprise at least one of a guiding element (1210), a handle (1201) and a mechanical stop intended to cooperate with a valve. The guiding element may be configured to cooperate with the reusable part of the system in such way the cartridge may be inserted into an opening of the reusable part and slide through it to a determined position. The mechanical stop may comprise a protrusion and planar surface intended to be on contact with a portion of tube such that a pinch valve may pinch the tube against the planar surface of the mechanical stop (1208).

### Pressure Sensor

Focusingd on FIGS. 29A and 29B, the pressure sensor (28) may comprise at least one of a pressure pod (1212) and a strain sensor (1300). The Strain sensor (1300) may be arranged in the reusable part while the pressure pod (1212) may be arranged in the cartridge. The strain sensor may be arranged to natively (by design) (at least during the treatment) apply a force against the pressure pod (e.g. a pressure pod membrane (1302)) in such a manner the strain sensor may measure positive and negative pressures.

The pressure pod (1212) may comprise at least one of a body (1301), a membrane (1302), and a membrane holder (1303). The membrane holder may be configured to hold the membrane (1302) against the body (1301). The body may comprise an opening which may be closed by the membrane. The membrane support may be further configured to hold the membrane with a force necessary to tightly closed the membrane to the body opening.

The pressure pod may further comprise at least one of a cavity arranged below the body opening, an inlet port (1305), and an outlet port (1305) in such a way that the fluid may enter through the inlet port, pass through the cavity, and exit through the outlet port. The pressure pod may be configured to fill at least partially the cavity and/or wet the membrane with the fluid whose pressure is to be measured.

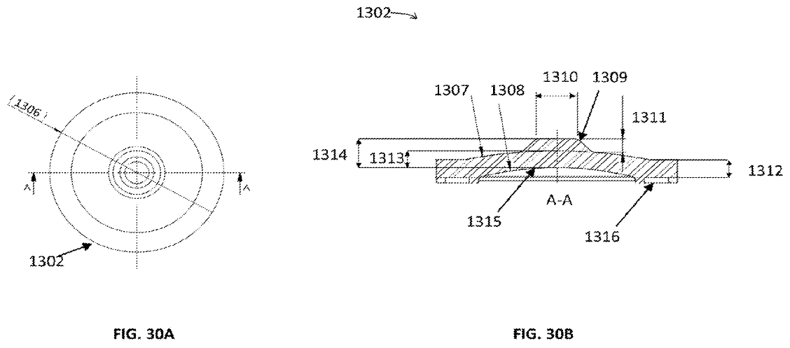

Focused on FIGS. 30A and 30B which show an embodiment of the membrane (1302), the membrane may have a circular shape with a radius (1306) which may be larger than the body opening. The membrane may have a dome chape. The inner wall (1315) of the membrane may form a concave cavity (with an inner bend radius (1307)) which may be wetted by the liquid (for example dialysate or blood). The membrane may further comprise a protrusion (1309) located to the center of the dome.

The membrane may be configured to homogeneously bend when a force is applied on the protrusion such that a pression in the cavity of the pressure pod is faithfully transmitted to the strain sensor (1300). The outer bend radius (1307) may be egal to or greater than the inner bend radius (1308) such that the thickness of the membrane center (1313) (for example without the protrusion thickness (1311)) is equal to or greater than the thickness of membrane edges (1312). The protrusion (1309) may provide an overall thickness at the center of the membrane larger than the thickness of membrane edges (1312).

The membrane may further comprise a groove (1316) around it edge to tightly closed the opening of body.

The membrane may comprise a versatile elastomer such as a liquid silicone rubber which may be mold. The membrane may be at least elastic or flexible. The hardness of membrane may be comprised between 30 and 100, preferentially between 280 and 100, more preferentially between 290 and 80 durometer Shore A.

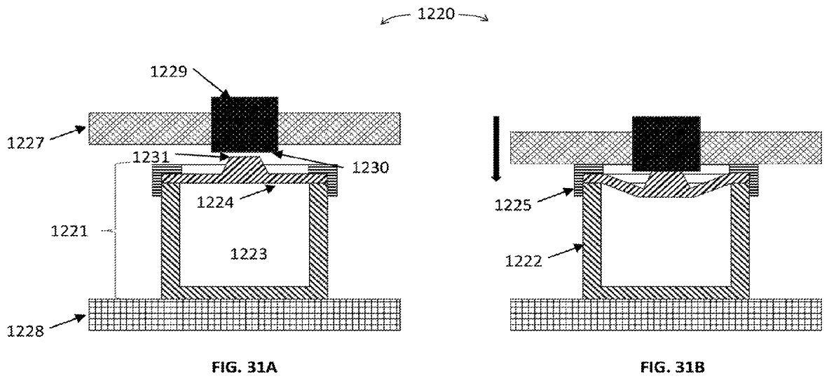

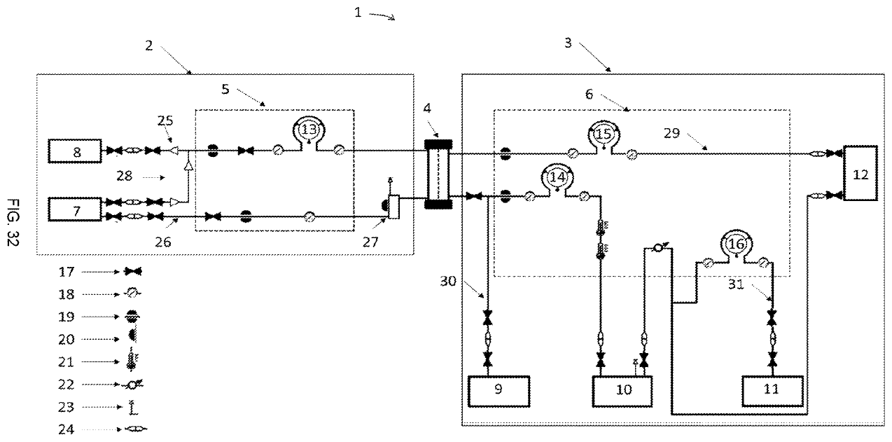

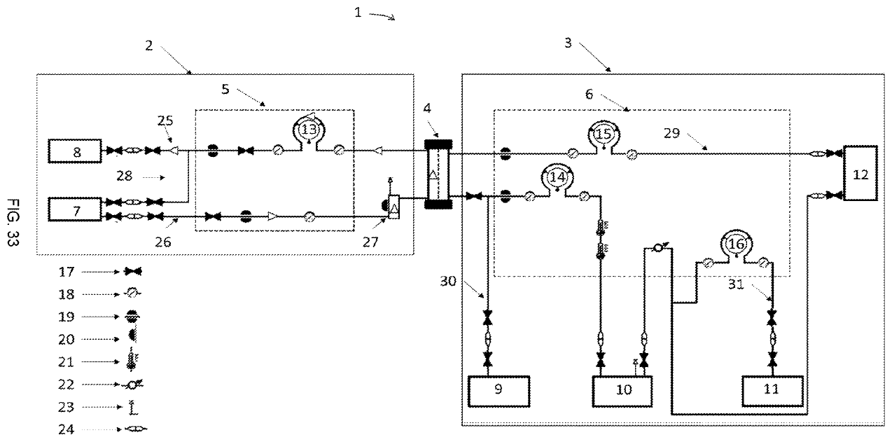

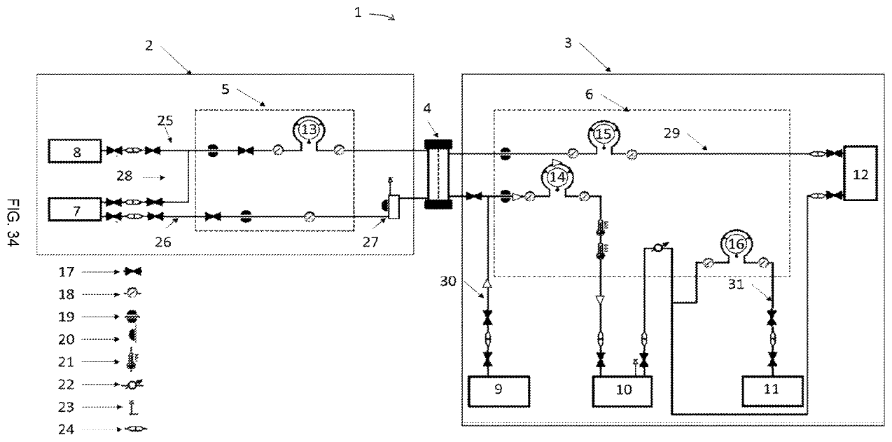

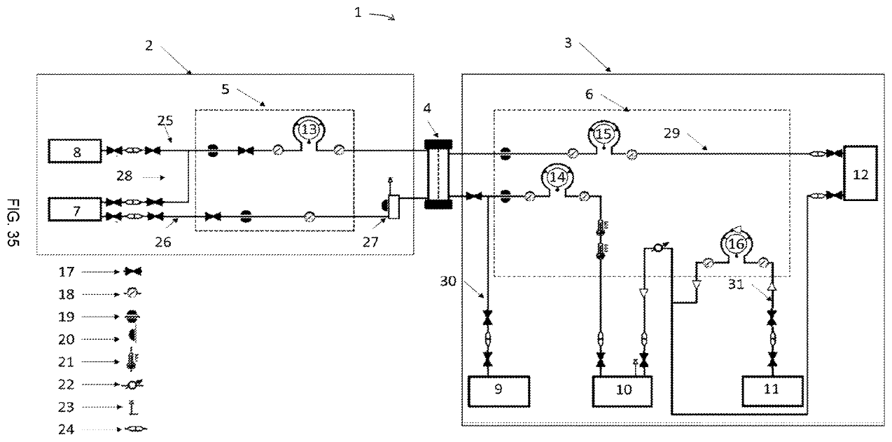

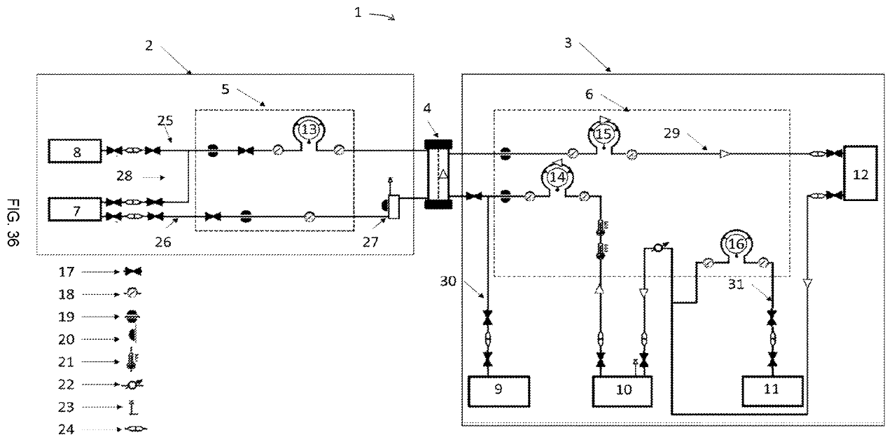

FIGS. 31A and 31B focus on a possible embodiment, the system (1220) may comprise:
A cartridge or a pressure pod or pressure sensor (1221) comprising a body (1222) with a cavity (1223) and an opening (1317 as shown by FIG. 29B), a flexible membrane (1224), at least one port (not show here), and a securing device or counter piece or ring (1225) configured to secure the flexible membrane to the body, and
An apparatus (1226) which may comprise:
A pressure transducer (1229) having a sensing portion (1230) configured to be removably coupled to the pressure sensor (1221),
A movable support (1227) on which the pressure transducer (1229) is secured and movable (e.g., along an axis), The apparatus may comprise at least one of a movable part (1227) (which may be or comprise the movable support) and a fixed part (1228). The movable part may move relatively to at least one of a cartridge, and fixed part.

The pressure sensor may comprise a fluid chamber defined by the cavity and the flexible membrane which closes the opening,

In one embodiment, when the flexible membrane is aligned with the axis, the pressure transducer may be configured to be moved toward the pressure sensor until the securing device makes contact with the movable support.

The sensing portion and the flexible membrane may be configured such that when the movable support and the securing device are in contact, the sensing portion exerts a force against the flexible membrane and measures a counterforce applied to the sensing portion by the flexible membrane and/or by the fluid within the cavity (e.g., the positive and/or the negative pressure).

The flexible membrane may further comprise a protrusion (1231) that extends away from the counter-piece/securing device.

The flexible membrane and the movable support may be configured in such a way that the flexible membrane makes contact with the sensing portion before the movable support makes contact with the securing device.

The apparatus may comprise a controller device which may be operatively coupled to the pressure transducer and/or a loading device. The movable support may be coupled to the loading device which drives the movable support. The loading device may comprise an electric motor and/or sensor which detect when the securing device is in contact with the movable support. For example, once the securing device has come into contact with the movable support the loading device may stop the driving.

### Leak Detector

Since a part of the fluid circuit may be insertable into the machine and enclosed within the machine during the dialysis operation, some of the fluid can flow and/or penetrate inside the machine (200) due to a leakage of the fluid circuit for example. In cases where leaks appear and go undetected in one of the system components, the leaking fluid can potentially damage the machine itself. Therefore, in one possible embodiment, the system may comprise a leak sensor.

In one possible embodiment, the machine may comprise at least one sensor configured to detect fluid leaks in the blood circuit and the dialysate circuit. Each fluidic circuit may have its own dedicated sensors, located at strategic spots underneath the respective disposable cartridge or fluid circuit.

The sensor may be based on capacitive touch technology. In this case, a dedicated integrated circuit (IC) detects changes in the capacitive field between one or several electrodes and a surrounding reference plane.

While this technology is normally used to detect finger touch, it can also serve as a general proximity or presence detector. The electrodes are incorporated as flat, printed copper features on a printed circuit board (PCB). Fluid drops bridging the gap between the electrodes and the surrounding reference plane will in fact change the capacitive field and thus be detectable to the sensor.

In order to trigger detection, a determined minimum quantity of fluid is necessary, depending on the size of the electrodes, the size of the gap with the surrounding plane, and on where fluid drops occur. Mechanical features (such as hole, fluid guiding means, groove, . . . ) have been implemented in the machine between the cartridges and the leak sensors to guide fluid leaks towards the electrodes. The sensitivity of the sensor can further be adjusted to suit the required detection capability.

The digital sensor outputs (“leak”, “no leak”) may be sampled at regular intervals by a controller device. In addition, a watch dog module (e.g., a heart-beat signal) allows the controller device to determine the correct operation of the leak sensor.

lt should be noted that the principle of detection is purely capacitive, not resistive. The electrodes and the reference plane may comprise a coating by a non-conductive lacquer (e.g., soldermask) and/or may comprise insulating layers that prevent corrosion when in contact with fluids. Therefore, fluid drops will not result in a galvanic connection between the electrodes and the reference plane.

### Priming Process

Before starting the treatment, the fluid circuit has to be primed to remove all gas initially stored in the fluid circuit.

To prime the blood circuit, the first bag (7) may initially store a priming solution (for example saline solution or a blood compatible solution), and the second bag (8) may initially be empty. The additional line may be primed by gravity without using the first pump (13) as shown by FIG. 32. In this case, the dedicated valve/clamp may be opened, and the priming solution may flow from the first bag (7) to at least the arterial line (25). The gas may flow to the second bag (initially empty). The first bag may be located above the connection between the additional line (28) and the arterial line (25). Once the additional line (28) is primed, the valve/clamp of this line can be closed.

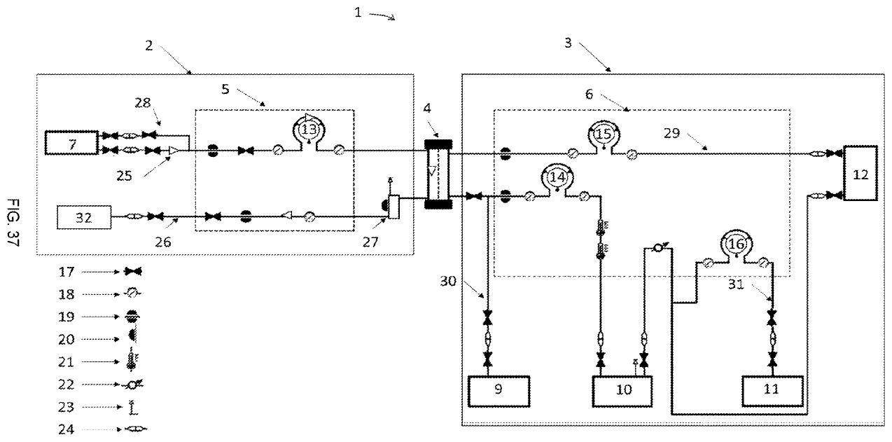

According to the embodiment shown by FIG. 33, to prime the remainder of the blood circuit, the first pump (13) may be actuated in reverse direction to move the priming solution from the first bag (7) to the second bag (8). During this step, the priming solution may fill the venous line, afterward the drip chamber (27), then the blood compartment of the first filter (4), and after the arterial line (25). The fluid (e.g. gas) initially present in the blood circuit is moved into the second bag (8). This step may be used to rinse/flush the blood circuit (2), to remove all manufacturing residues form the blood circuit.

This step may be launched by the user or the processor and may be stopped by the processor. The processor may monitor the smooth operating of the priming process by using at least one of a pressure sensor (18), a level sensor (20), an air sensor (19), and a sensor monitoring the first pump (13). The processor may stop the priming process depending on the measurements of these sensors for example if the pressure measured reaches a determined threshold or does not reach another determined threshold for a determined period of time.

The priming of the blood circuit may be stopped by the processor due to a failure, the end of the process, the end of the process and/or after a determined time period (during which the first pump runs), a number of actuations of the first pump, a number of revolutions of the first pump (if the first pump is a peristaltic pump). If no failures occur during this process (e.g. for a determined time period), the processor may stop the first pump and/or may determine that the blood circuit is fully primed.

The second bag (which is initially empty) may be configured to collect at least one of the initial fluid (gas) and a portion of the volume of the priming solution used to rinse/flush the blood circuit.

With this configuration, the user has no need to manipulate the first filter (e.g. dialyzer) and/or the drip chamber to achieve proper priming. During this phase, several functional tests may be executed such as pressure sensor and pump tests.

Standard dialysate solutions may contain HCO3 (bicarbonate) and Ca/Mg during the treatment. This combination cannot be stored though, as Ca and/or Mg-carbonate would precipitate. Furthermore, solutions containing HCO3 and glucose together cannot be steam sterilized as the high pH value would lead to the formation of harmful glucose degradation products. Prior art systems therefore have to use double compartment bags, where an acidic solution containing Ca, Mg and glucose is physically separated from an alkaline solution containing HCO3. This is cumbersome, costly, and prone to user error. Mixtures of only NaCl and NaHCO3 are easy to prepare, easy to store, and less expensive.

The third bag (9) may initially store an initial solution. The third bag may be configured to store a volume of fluid comprised between 0.5 L and 4 L, preferentially between 1 L and 3 L, for example 2 L. In one embodiment, the third bag may comprise a single storing compartment which store the initial solution. This initial solution may be not a dialysate solution ready for use (for the treatment), for example, the initial solution may comprise at least one of water, sodium (for example sodium chloride NaCI) and bicarbonate (for example sodium bicarbonate NaHCO3) but the initial solution may not comprise others electrolytes (such as for example at least one of magnesium, calcium and potassium).

In one embodiment, the initial solution may comprise at least one of sodium chloride (NaCl), sodium bicarbonate (NaHCO3) and sodium carbonate (Na2CO3). The initial solution may comprise 0-200 mmol/L NaCl, 0-200 mmol/L NaHCO3 and/or 0-100 mmol/L Na2CO3; preferentially: 100-140 mmol NaCl, 40-80 mmol/NaHCO3 and/or 20-40 mmol/L Na2CO3; for example: 110 mmol/L NaCl and/or 37 mmol/L NaHCO3

The initial solution may not comprise Calcium (Ca), Magnesium (Mg), Potassium (K) or glucose.

In one embodiment, the fourth bag may be initially empty and may have to be filled with the initial solution prior to beginning treatment. In another embodiment, the system may not comprise the third bag. In one embodiment, the fourth bag may initially store the initial solution as described above and may comprise a single storing compartment.

FIG. 34 illustrates a transfer process for filling the fourth bag (10) with the solution initially stored in the third bag (9). The valve/clamp dedicated may be opened while the others may be closed. The second pump may be operated to move the initial solution from the third bag (9) to the fourth bag (10). The second pump may be operated in a reverse direction during the transfer process while during the treatment the second pump (14) may be operated in a normal direction. The reverse direction may be opposite to the normal direction.

This step may be launched by the user or the processor and may be stopped by the processor. The processor may monitor the smooth operating of the transfer process by using at least one of a pressure sensor (18), a weighing scale (which weighs the fourth bag (10)) and a sensor monitoring the second pump (14). The processor may stop the transfer process depending on the measurements of these sensors for example if the measurement reaches a determined threshold or does not reach another determined threshold for a determined period of time. The processor may monitor the weight of the fourth bag (10) for the time period of the transfer process.

The transfer process may be stopped by the processor due to a failure, the end of the process and/or after a determined time period (during which the second pump runs), a number of actuations of the second pump, a number of revolutions of the second pump (if the second pump is a peristaltic pump), if the pressure measurement sharply dips (in case of the third bag (9) being empty), if the weight of the fourth bag reaches a determined threshold. If no failures occur during this process (e.g. for a determined time period), the processor may stop the second pump and/or may determine that the transfer process is completed.

In the case where the third bag (9) is located above the fourth bag (10), the transfer process may be (also or a partly) performed by gravity. The transfer process may be used as part of the dialysate priming process. In one embodiment, the fourth bag (10) is placed on a warmer which may comprise a weighing scale. During or after the end of the transfer process, the warmer may heat the initial solution in the fourth bag (10).

In some possible embodiments, after the end of the transfer process, the rest of the dialysate priming process can begin.

In one embodiment, at least a part of the transfer process may be processed in same time of the blood priming sequence. In one embodiment, at least a part of the dialysate priming process may be processed in same time of the blood priming sequence.

FIG. 35 illustrates the second supply line (31) priming process. The fifth bag (11) may comprise an additive solution to be added to the solution circulating in the dialysate loop line (29) required for the treatment (due to the effect of the second filter (e.g., sorbent)). The additive solution may comprise water and electrolyte(s) (such as for example at least one of magnesium, calcium, and potassium) but the additive solution may not comprise bicarbonate (for example sodium bicarbonate). The additive solution may be added to the dialysate loop line (29) between the second filter (12) and the fourth bag (10) for example upstream of the fourth bag (10) and/or downstream of the second filter (12). The dedicated valve/clamp may be opened while the others may be closed. The fourth pump (16) may be operated to move the additive solution from the fifth bag (11) to the fourth bag (10).

This step may be launched by the user or the processor and may be stopped by the processor. The processor may monitor the smooth operating of this process by using at least one of a pressure sensor (18), a weighing scale (which weighs the fourth bag (10)) and a sensor monitoring the fourth pump (16). The processor may stop this process depending on the measurements of these sensors for example if a measurement reaches a determined threshold or does not reach another determined threshold for a determined period of time. The processor may monitor the weight of the fourth bag (10) for the time period of this process.

This process may be stopped by the processor due to a failure, the end of the process and/or after a determined time period (during which the fourth pump runs), a number of actuations of the fourth pump, a number of revolutions of the fourth pump (if the fourth pump is a peristaltic pump), if the weight of the fourth bag reaches a determined threshold or increases. If no failures occur during this process (e.g. for a determined time period), the processor may stop the fourth pump and/or may determine that this process is completed.

Since the weight of the fourth bag (10) may be monitored by the weighing scale, a functional test of the fourth pump may be performed during the additive line priming process. The fourth pump may be first operated to prime the additive line (in some cases a part of the dialysate loop line). Afterwards, when the additive solution reaches the fourth bag (10), the weight of the fourth bag begins to increase, and the fourth pump (16) may be operated to perform a functional test based on the weigh scale measurements. The functional test of the fourth pump may be performed after the priming process of the overall dialysate circuit (for example during the running-in of the sorbent as described below because even if the second and the third pump are operated, the weight of the fourth bag does not fluctuate and thus the functional test of the fourth pump may be performed using the weighing scale of the fourth bag). The processor may monitor the weigh scale measurements to determine the smooth operating of the fourth pump and/or to stop the second supply line (31) priming process.

FIG. 36 illustrates the dialysate priming process. This process may be performed before, during or after the additive line priming process. This process may allow at least to prime the dialysate loop line (29), to run in the second filter (12) and/or to prepare the dialysate solution for the treatment.

During this process, the second pump (14) and the third pump (15) are operated to move the solution stored in the fourth bag (10) into the dialysate circuit (for example in the line (29)), pushing the initially contained fluid (e.g., gas), in the dialysate compartment of the first filter (4) and in the second filter) into the fourth bag (10). The fourth bag (10) may comprise an air flow device (23) configured to expel the gas to the exterior. The air flow device (23) may comprise a hydrophobic membrane configured to allow the passage of gas but not the liquid. Therefore, any air or other gas within the bag can pass the air flow device while the liquid cannot pass the air flow device and will remain inside the fourth bag (10).

To prime the dialysate loop line (29), the solution stored (which is preferentially not ready for use) is moved through the line and reaches successively the second pump, the first filter, the third pump, the second filter and then back into the fourth bag. The processor may be configured to monitor the weighing scale of the fourth bag (10). During this priming process the weight of the fourth bag may initially decrease and then remain stagnant (level off). The processor may be configured to monitor the priming process by using the measurements of the weighing scale. When the weight starts to remain stagnant, (i.e. the weight is leveling off), the processor may be configured to determine that the dialysate loop line is fully primed. In the case where the fourth pump (16) is also operating, the processor may take into account the additive solution added into the fourth bag to determine when the dialysate loop line is fully primed.

The second filter may be a sorbent configured to remove toxins from spent dialysate (after passing through the first filter (for example the dialyzer)). For the sorbent to work properly (optimally or efficiently), the sorbent may be run in to allow the chemical elements that make it up to be fully effective. Therefore, even if the dialysate line is fully primed the second pump and the third pump may continue to operate to pass solution through the sorbent for a determined time period in order to run in the second filter.

To start the dialysis treatment, the solution stored in the fourth bag (10) must be a dialysate solution, and the initial solution cannot be used as dialysate solution because the initial solution does not comprise the required electrolytes. To prepare the dialysate solution, additive solution must be added to the initial solution before the treatment is started. In addition, during dialysate regeneration, the sorbent removes the electrolytes Ca, Mg and K from spent dialysate, and the additive solution is therefore also added to the dialysate loop line throughout the treatment. In other words, the additive solution may be added before the treatment is started to prepare the dialysate solution and over the treatment to regenerate the dialysate solution.

Therefore, in one embodiment, during the dialysate circuit priming process, the initial solution may be moved by the second pump and the third pump through the dialysate loop line and the additive solution may be added to the initial solution to the fourth bag (e.g., downstream of the second filter and upstream of the fourth bag) to get a mixing solution until to reach a dialysate solution required to the treatment. With this process, the dialysate loop line, the dialysate compartment of the first filter, and the second filter are primed, the second filter is run in, and the dialysate solution is prepared gradually over this process.

This step may be launched by the user or the processor and may be stopped by the processor. The processor may monitor the smooth operating of this process by using at least one of a pressure sensor (18), a weighing scale (which weighs the fourth bag (10)) and a sensor monitoring the second, third, and fourth pump (14, 15, 16). The processor may stop this process depending on the measurements of these sensors for example if a measurement reaches a determined threshold or does not reach another determined threshold for a determined period of time. The processor may monitor the weight of the fourth bag (10) for the time period of this process.

This process may be stopped by the processor due to a failure, the end of the process and/or after a determined time period (during which at least one of the second, third, and fourth pump run(s)), a number of actuations of the pump(s), a number of revolutions of the pump(s) (if the pump is a peristaltic pump), depending on the weight of the fourth bag or its change. If no failures occur during this process (e.g. for a determined time period), the processor may stop the pumps and/or may determine that this process is completed.

Once all circuits have been fully primed, the second bag (8) may be disconnected from the arterial line (25) and the first bag (7) may be disconnected form the venous line (26). Alternatively, the first bag (7) may stay connected (or may be replaced by another bag (e.g. a saline bag) which may be used to infuse fluid in case of a drop in the patient's blood pressure and/or for the blood return process). In one embodiment, the first pump may be operated again in the reverse direction prior to disconnection of the bags from the arterial line and the venous line, to flush again and/or to remove last remnants of waste or air bubbles present in the blood circuit. Afterward, the arterial line and the venous line can be connected to the patient (32).

FIG. 37 illustrates the blood return process. For example, at the end of the treatment, the blood present in the blood circuit must be returned to the patient (32). The patient is disconnected from the arterial line and the arterial line is connected to the second bag which stores the remaining solution from priming, or to another bag of fresh fluid, e.g. saline. The first pump (13) is actuated in normal direction to move the blood back to the patient pushed by the remaining priming solution or saline.

### Draining Process

According to one embodiment, the system may be configured to carry out a hemodialysis treatment at home. In order to limit the volume of dialysate required for the treatment, the system may be configured to recirculate the dialysate solution over the treatment. In this case, once the dialysate solution has passed through the dialyzer, the dialysate solution is deemed spent and must be cleaned with a sorbent device and electrolytes must be added to the cleaned dialysate before passing through the dialyzer.

In some embodiment, during the hemodialysis treatment, the liquid removed from the patient by ultrafiltration through the dialyzer, the additive solution (electrolyte) and the dialysate are collected in a single bag (also called weighing bag or fourth bag thereafter). At the beginning, the weighing bag is filled with a volume of 2 L but at the end of a 2 h dialysis session, the weighing bag is filled with a volume up to 5 L of liquid.

At the end of the treatment, the user must detach the weighing bag and lift it out of the machine. It could be difficult for elderly patient or people with a disability, to lift a weight of approximatively 5 kg. The aim of the present disclosure is to help the patient during the disposable set removal phase and avoid him to lift too much weight.

As disclosed above, FIG. 34 illustrates a transfer process for filling the fourth bag (10) with the solution initially stored in the third bag (9). The valve/clamp dedicated may be opened while the others may be closed. The second pump may be operated to move the initial solution from the third bag (9) to the fourth bag (10). The second pump may be operated in a reverse direction during the transfer process while during the treatment the second pump (14) may be operated in a normal direction. The reverse direction may be opposite to the normal direction.

The fifth bag (11) may initially store an additive solution which may comprise electrolyte required to reconstitute the dialysate solution. The fifth bag may be configured to store a volume of fluid comprised between 0.5 L and 5 L, preferentially between 1 L and 4 L, for example 2.5 L. The volume of this bag may depend on the time duration of the treatment and on the concentration of the electrolyte. The additive solution may comprise water and electrolyte (such as for example at least one of magnesium, calcium, and potassium).

During the treatment, the second pump (14) and the third pump (15) may be operated to move the dialysate through the dialysate loop line and the fourth pump (16) may be also operated to inject the additive solution into the dialysate loop line (29). The additive solution may be added to the dialysate loop line (29) between the second filter (12) and the fourth bag (10) for example upstream of the fourth bag (10) and/or downstream of the second filter (12).

At the end of the treatment, the fourth bag may comprise the initial volume of the dialysate, the volume of additive solution added and the UF due to the treatment and the volume of the fourth bag may amount to 5 L. The system may be configured to drain at least a part of the fluid present in the fluid circuit. The system may be configured to drain at least a part of the fluid volume stored in the fourth bag (10) through at least one of a supply line (30, 31). For example, at the end of the treatment, once the patient is no longer connected to the fluid circuit.

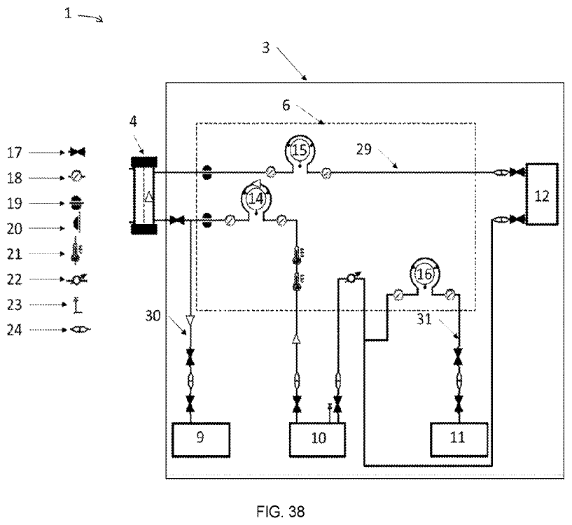

As disclosed by FIG. 38, the system may be configured to drain the fluid to the third bag (9). In this case, the processor may be configured to operate the second pump (14) to move a volume of the solution from the fourth bag (10) to the third bag (9). The processor may monitor at least one of the pressure sensors around the second pump, the weighing scale of the fourth bag. The draining process may be launched by the user or by the processor. The processor may stop the process automatically after a determined time duration, after a number of actuations of the second pump, a number of revolutions of the second pump (if the second pump is a peristaltic pump) or depending on the measurements of the weighing scale of the fourth bag.

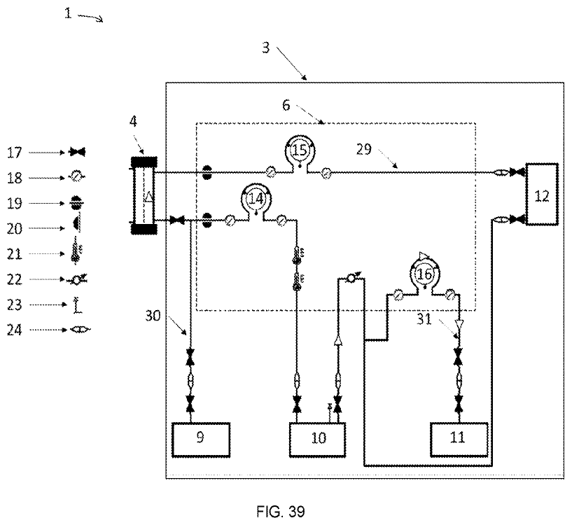

As disclosed by FIG. 39, the system may be configured to drain the fluid to the fifth bag (11). In this case, the processor may be configured to operate the fourth pump (16) (in a reverse direction which is opposite to the normal direction used during the treatment) to move a volume of the solution from the fourth bag (10) to the fifth bag. The processor may monitor at least one of the pressure sensors around the fourth pump, the weighing scale of the fourth bag. The draining process may be launched by the user or by the processor. The processor may stop the process automatically after a determined time duration, after a number of actuations of the fourth pump, a number of revolutions of the fourth pump (if the fourth pump is a peristaltic pump) or depending on the measurements of the weighing scale of the fourth bag.

The system may use one or both of the above-described emptying processes simultaneously or sequentially. With this solution, the volume initially stored in the fourth bag at the end of treatment may be shared between at least two of the third, fourth and fifth bags (9, 10, 11).

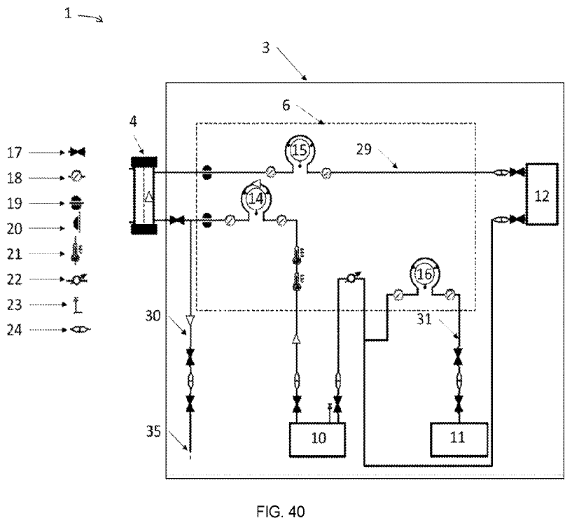

As disclosed by FIG. 40, the system may be configured to drain the fluid to a sewer of a home (e.g. in a sink) via a drain line (35) removably connected to the first supply line (30). The third bag may be disconnected from the first supply bag and the drain line (35) may be connected to the first supply line (30) (for example instead of the third bag). The processor may be configured to operate the second pump (14) to move a volume of the solution from the fourth bag (10) through the first supply line (30) and then through the drain line (35). The processor may monitor at least one of the pressure sensors around the second pump, the weighing scale of the fourth bag. The draining process may be launched by the user or by the processor. The processor may stop the process automatically after a determined time duration, after a number of actuations of the second pump, a number of revolutions of the second pump (if the second pump is a peristaltic pump) or depending on the measurements of the weighing scale of the fourth bag.

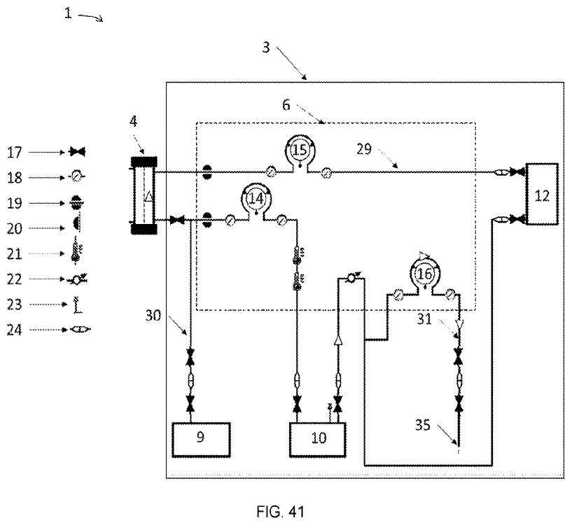

As disclosed by FIG. 41, the system may be configured to drain the fluid to a sewer of a home (e.g. in a sink) via a drain line (35) removably connected to the second supply line (31). The fifth bag may be disconnected from the second supply bag and the drain line (35) may be connected to the second supply line (31) (for example instead of the fifth bag). The processor may be configured to operate the fourth pump (16) (in a reverse direction which is opposite to the normal direction used during the treatment) to move a volume of the solution from the fourth bag (10) through the second supply line (31) and then through the drain line (35). The processor may monitor at least one of the pressure sensors around the fourth pump, the weighing scale of the fourth bag. The draining process may be launched by the user or by the processor. The processor may stop the process automatically after a determined time duration, after a number of actuations of the fifth pump, a number of revolutions of the fourth pump (if the fourth pump is a peristaltic pump) or depending on the measurements of the weighing scale of the fourth bag.

One skilled in the art will understand that various combinations and/or modifications and variations can be made in the described systems and methods depending upon the specific needs for operation. Moreover features illustrated or described as being part of an aspect of the invention may be used in the aspect of the invention, either alone or in combination.

1.-118. (canceled)

119. A system comprising:
a container comprising:
flexible walls defining a variable volume,
an inlet tube intended to feed the container with a liquid solution and/or gas, and
an outlet tube intended to allow the liquid solution to flow out of the container,
a weighing device comprising a tray configured to support the container thereon during use, and
a securing device coupled to the tray configured to secure the container to the tray and/or to limit deformation of at least one of flexible walls such that the container cannot induce a disturbance in the weighing of the container.

120. The system according to claim 119, the outlet tube is configured to extend through the inner compartment of the container.

121. The system according to claim 120, wherein the container further comprises a lower surface on which at least a part of the outlet tube is secured.

122. The system according to the claim 121, wherein an end of the outlet tube is secured on the lower surface of the container.

123. The system according to claim 120, wherein the length of the outlet tube extending through the inner compartment is less than one third of the length of the inner compartment of the container.

124. The system according to claim 120, wherein the outlet tube is configured to extend further into the inner compartment than the inlet tube.

125. The system according to claim 120, wherein the container further comprises a vent device.

126. The system according to the claim 125, wherein the vent device comprises a hydrophobic material configured to allow the passage of gas but not liquid.

127. The system according to the claim 125, wherein the vent device is arranged on a top wall of the container such that the air present in the inner compartment of the container is natively in contact with the vent device.

128. The system according to the claim 127, wherein the vent device is arranged at the middle of the top wall.

129. The system according to claim 120, wherein the securing device comprise at least one of a hook, a strap, a flange, and a magnetic attachment.

130. The system according to claim 120 further comprising a cavity having a lower inner surface and an upper inner surface opposite to the lower inner surface.

131. The system according to the claim 120, wherein the container is intended to be placed between the lower inner surface and the upper inner surface in use and the securing device is configured to prevent any contact of the container to the upper inner surface.

132. The system according to claim 120, wherein the securing device is configured to secure at least two opposite ends of the container to the tray.
